(19) United States
(12) Patent Application Publication
(10) Pub. No.: US 2020/0365256 A1
(43) Pub. Date: Nov. 19, 2020

(54) PATIENT STATUS DETERMINATION DEVICE, PATIENT STATUS DETERMINATION SYSTEM, PATIENT STATUS DETERMINATION METHOD, AND PATIENT STATUS DETERMINATION PROGRAM RECORDING MEDIUM

(71) Applicant: NEC CORPORATION, Tokyo (JP)

(72) Inventors: Masahiro HAYASHITANI, Tokyo (JP); Yuan LUO, Tokyo (JP); Masahiro KUBO, Tokyo (JP); Shigemi KITAHARA, Tokyo (JP)

(73) Assignee: NEC CORPORATION, Tokyo (JP)

(21) Appl. No.: 16/770,347

(22) PCT Filed: Nov. 6, 2018
(86) PCT No.: PCT/JP2018/041155

§ 371 Date: Jun. 5, 2020

(30) Foreign Application Priority Data
Dec. 8, 2017 (JP) 2017-235687

(51) Int. Cl. G16H 40/20; G16H 20/00; G10L 15/02

(52) U.S. Cl. CPC G16H 40/20; G10L 15/02; G16H 20/00

(57) ABSTRACT

The present invention addresses the problem of improving a task of a medical staff person. A patient status determination device includes: an indication unit that indicates to the medical staff person, in order, a plurality of items to be measured for the patient; a reaction recognition unit that recognizes a reaction from the medical staff person to the indicated items, and outputs the recognized result; and a score determination unit that determines the score for the indicated items on the basis of the recognized result, and outputs the determined score. The indication unit indicates to the medical staff person each of the plurality of items in the form of at least one question associated with the items.

### TECHNICAL FIELD

The present invention relates to a patient status determination device, a patient status determination system, a patient status determination method, and a patient status determination program recording medium.

### BACKGROUND ART

In a hospital (especially, an acute care hospital), a situation where a person engaged in medical care (will be called a “medical staff person” hereinafter), such as a doctor or a nurse, is required to measure (determine) a status of a patient emergently transported thereto by an ambulance or the like takes place frequently. It may be presumed that the patient emergently transported is suspected to have a cerebrovascular disorder. In such a circumstance, the medical staff person is required to understand the status (e.g. paralysis, ataxia or the like) of the patient.

Measurement techniques for measuring (determining) the status of the patient suspected to have the cerebrovascular disorder have been known in this technical field. As one of the measurement techniques, there is the NIHSS (National Institute of Health Stroke Scale). In the NIHSS, on arrival of the patient at the hospital, before a treatment, and during the treatment, it is necessary for the medical staff person in charge to measure (determine) the status of the patient in question in every measurement period of fifteen minutes at the shortest. This is because the status of the patient is concerned in a treatment plan of the patient. It has been known that the NIHSS has thirteen items as items to be measured (determined). Accordingly, the medical staff person is required to perform a task (operation) of selecting a score for each item of the NIHSS within a measurement time interval (determination time interval) in every measurement period mentioned above. A total sum of scores of the respective items is equal to forty-two. In addition, the measurement time interval (determination time interval) is desirably as short as possible as compared with the above-mentioned measurement period and, for example, is preferably three minutes.

Various techniques related to this invention have been proposed.

For example, Patent Literature 1 discloses a system for classifying one or more subjects (patients) into one or more categories indicative of a health condition associated with the one or more subjects (patients). Patent Literature 1 describes that a health condition score may correspond to a stroke score such as a NIHSS score. Patent Literature 1 describes an example in which the NIHSS score is classified into four categories and then a doctor determines the degree of severity of the stroke based on the category into which the subject has been classified.

Patent Literature 2 discloses a remote medical system in which a diagnosis for a patient is performed by a doctor in a remote place. Patent Literature 2 describes, as one example of the remote medical system, a system which measures a body sound of a patient (subject to be measured) by using an electronic stethoscope at a medical site and records acquired electronic data, namely, body sound information. Such a remote medical system allows a diagnosing person such as a doctor in a remote place from the medical site to refer to the body sound information to use it for the diagnosis for the patient. In a measurement assistance device disclosed in Patent Literature 2, a display unit displays information (measurement scenario) for assisting a measurement activity of a measuring person and displays an operation screen as a GUI (Graphical User Interface) screen. Patent Literature 2 illustrates one example of a measurement assistance screen in which contents of the measurement scenario are reflected. The measurement assistance screen has an output area in which an auscultation procedure included in the measurement scenario is displayed, and an output area in which auscultation region visualization information is displayed. The measuring person confirms image data and starts auscultation in accordance with the measurement assistance screen. When acquisition of the body sound of the patient finishes, the measuring person taps a button.

### CITATION LIST

### Patent Literatures

PL 1: JP 2016-157430 A

PL 2: JP 2014-023715 A

### SUMMARY OF INVENTION

### Technical Problem

In the above-mentioned NIHSS, the medical staff person (especially, an untrained and inexperienced medical staff person) may hesitate to determine a selection of a score for each item. As a result, it may possibly take a long time to measure (determine) the NIHSS. Therefore, it is desirable to resolve a problem of such hesitation of the medical staff person in decision on measurement (determination) and to shorten the measurement time interval (determination time interval).

However, the above-mentioned Patent Literatures 1 and 2 cannot resolve such problems.

That is, Patent Literature 1 merely discloses a technical idea of classifying a range of the NIHSS score into a plurality of categories. Accordingly, in Patent Literature 1, the medical staff person must, as ever, perform the above-mentioned task (operation) of selecting the score for each item of the NIHSS. Therefore, it is not possible to resolve the problems of the hesitation in decision on the measurement (determination) and of the measurement time interval (determination time interval).

On the other hand, Patent Literature 2 merely discloses the remote medical system in which the measuring person carries out the measurement (auscultation) for the patient and the doctor in the remote place performs the diagnosis by using a result of auscultation. In such a remote medical system, the measuring person merely performs an appropriate measurement for the patient by using a measurement instrument, such as the electronic stethoscope, in accordance with the measurement scenario. As described above, in the NIHSS, it is essential not only to perform the measurement but also to select the score for each item. Therefore, in the remote medical system in Patent Literature 2 also, the medical staff person as the measuring person, as ever, hesitates to determine selection of the score. Accordingly, in Patent Literature 2 also, it is impossible to resolve the problems of the hesitation in decision on the measurement (determination) and of the measurement time interval (determination time interval).

It is an object of the present invention to resolve the above-mentioned problems and to provide a patient status determination device, a patient status determination system, a patient status determination method, and a patient status determination program recording medium, which are capable of improving an operation of a medical staff person.

### Solution to Problem

A patient status determination device according to the present invention comprises an indication unit configured to sequentially indicate, to a medical staff person, a plurality of items to be measured for a patient; a reaction recognition unit configured to recognize a reaction from the medical staff person to the indicated item to produce a recognized result; and a score determination unit configured to determine, based on the recognized result, a score for the indicated item to produce a determined score, wherein the indication unit is configured to present, to the medical staff person, each of the plurality of items in the form of at least one question associated with the item.

A patient status determination system according to the present invention comprises the patient status determination device described above and a totalization device configured to receive the determined score from the patient status determination device to totalize the score.

A patient status determination method according to the present invention comprises sequentially indicating, by an indication unit, to a medical staff person, a plurality of items to be measured for a patient; recognizing, by a reaction recognition unit, a reaction from the medical staff person to the indicated item to produce a recognized result; and determining, by a score determination unit, based on the recognized result, a score for the indicated item to produce a determined score, wherein the indication unit is configured to present, to the medical staff person, each of the plurality of items in the form of at least one question associated with the item.

A patient status determination program recording medium according to the present invention is a recording medium recording a patient status determination program which causes a computer to execute an indication step of sequentially indicating, to a medical staff person, a plurality of items to be measured for a patient; a reaction recognition step of recognizing a reaction from the medical staff person to the indicated item to produce a recognized result; and a score determination step of determining, based on the recognized result, a score for the indicated item to produce a determined score, wherein the indication step causes the computer to present, to the medical staff person, each of the plurality of items in the form of at least one question associated with the item.

### Advantageous Effect of the Invention

According to the present invention, it is possible to improve an operation of a medical staff person.

### BRIEF DESCRIPTION OF THE DRAWINGS

FIG. 1 is a block diagram for illustrating a configuration of a patient status determination system according to a first example embodiment of the present invention;

FIG. 2 is a flow chart for illustrating a flow of operation of a patient status determination device illustrated in FIG. 1;

FIG. 3 is a block diagram for illustrating a configuration of a patient status determination system according to a second example embodiment of the present invention;

FIG. 4 is a block diagram for illustrating a configuration of a patient status determination system according to a third example embodiment of the present invention;

FIG. 5 is a flow chart for illustrating a flow of operation of a patient status determination device illustrated in FIG. 4;

FIG. 6 is a flow chart for describing an operation of changing, in the patient status determination device illustrated in FIG. 4, contents of notification to be indicated by an indication unit by giving feedback before starting measurement;

FIG. 7 is a block diagram for illustrating a configuration of a patient status determination system according to a fourth example embodiment of the present invention;

FIG. 8 is a flow chart for illustrating a flow of operation of a patient status determination device illustrated in FIG. 7;

FIG. 9 is a view for illustrating thirteen items of the NIHSS;

FIG. 10 is a flow chart for illustrating guidance and questions which are indicated by the indication unit for an item of “1a. Level of Consciousness”;

FIG. 11 is a flow chart for illustrating guidance and a question which are indicated by the indication unit for an item of “1b. Disturbance of Consciousness—Question”;

FIG. 12 is a flow chart for illustrating guidance and a question which are indicated by the indication unit for an item of “1c. Disturbance of Consciousness—Response to Verbal Command”;

FIG. 13 is a flow chart for illustrating guidance and questions which are indicated by the indication unit for an item of “2. Best Gaze”;

FIG. 14 is a flow chart for illustrating guidance and a question which are indicated by the indication unit for an item of “3. Visual Field” and an example of display on a display screen of a display device with a touch panel;

FIG. 15 is a flow chart for illustrating guidance and questions which are indicated by the indication unit for an item of “4. Facial Palsy”;

FIG. 16 is a flow chart for illustrating guidance and questions which are indicated by the indication unit for an item of “5. Movement of Arms”;

FIG. 17 is a flow chart for illustrating guidance and questions which are indicated by the indication unit for an item of “6. Movement of Legs”;

FIG. 18 is a flow chart for illustrating guidance and a question which are indicated by the indication unit for an item of “7. Motor Ataxia” and an example of display on a display screen of a display device with a touch panel;

FIG. 19 is a flow chart for illustrating guidance and questions which are indicated by the indication unit for an item of “8. Sensory”;

FIG. 20 is a flow chart for illustrating guidance and questions which are indicated by the indication unit for an item of “9. Best Language”;

FIG. 21 is a flow chart for illustrating guidance and questions which are indicated by the indication unit for an item of “10. Dysarthria”; and

FIG. 22 is a flow chart for illustrating guidance and questions which are indicated by the indication unit for an item of “11. Extinction and Inattention.”

### DESCRIPTION OF EMBODIMENTS

Now, example embodiments of the present invention will be described in detail with reference to the drawings. Note that, in respective figures, the same or the corresponding parts are assigned with the same symbols and description of overlapping portions will be omitted as appropriate.

### First Example Embodiment

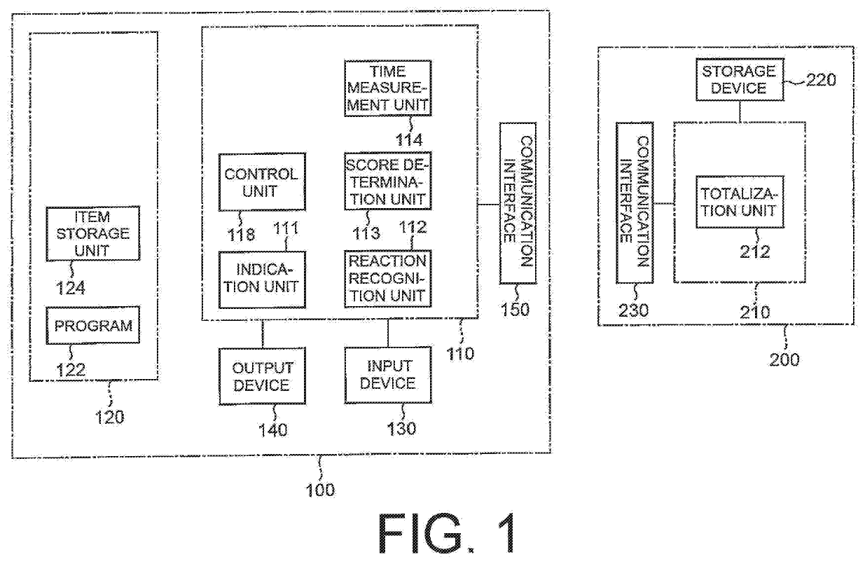

FIG. 1 is a block diagram for illustrating a configuration of a patient status determination system according to a first example embodiment of the present invention. As illustrated in FIG. 1, the patient status determination system comprises a patient status determination device 100 and a totalization device 200. The patient status determination device 100 and the totalization device 200 may be on the same device.

In the first example embodiment, the following case is supposed. In the illustrated patient status determination system, it is supposed that a medical staff person such as a nurse or a doctor measures (determines) a patient status in real time in every measurement period. In this case, the patient may be, for example, a patient emergently transported by an ambulance or the like. Such a patient is suspected to have a disorder such as a cerebrovascular disorder. The measurement period is, for example, fifteen minutes at the shortest. The patient status is, for example, paralysis, ataxia, and so on. In every measurement period, it is necessary for the medical staff person to perform a task of selecting, within a measurement time interval (determination time interval), a score for each of a plurality of items on the patient status. The plurality of items may be thirteen items which are defined by the above-mentioned NIHSS. The thirteen items defined by the NIHSS will later be described in detail with reference to the drawing. The measurement time interval (determination time interval) is desirably as short as possible as compared with the above-mentioned measurement period and is, for example, preferably three minutes.

The patient status determination device 100 is a device which can automatically determine the patient status in real time at a medical site. It is assumed that a patient ID (identifier) is assigned to the patient. Furthermore, a staff ID (user ID) may be assigned to the medical staff person.

The patient status determination device 100 comprises a data processing device 110 for processing data, a storage device 120 for storing a program and data, which will later be described, an input device 130 for inputting the data, an output device 140 for outputting the data, and a communication interface 150. Such a patient status determination device 100 may be implemented by, for example, a tablet PC (personal computer).

The output device 140 comprises a display device such as a LCD (Liquid Crystal Display) or a PDP (Plasma Display Panel), a printer, and a receiving set. The receiving set may be an earphone or a headphone. The output device 140 has a function of displaying, on a screen of the display device, a variety of information such as an operation menu and a plurality of questions associated with respective items, which will later be described, of informing each of the plurality of questions by a speech from the receiving set, and of printing a final result on the printer.

The storage device 120 is a storage medium which can hold a variety of data. For instance, the storage device 120 comprises the storage medium such as a HDD (Hard Disk Drive) or a SSD (Solid State Drive) or a memory such as a ROM (read only memory) and a RAM (random access memory). The storage device 120 has a function of storing a program 122 and processing information (which will later be described) required for a variety of processing in the data processing device 110.

The data processing device 110 comprises a microprocessor such as a MPU (micro processing unit) or a CPU (central processing unit). The data processing device 110 has a function of reading the program 122 from the storage device 120 to implement various processing units for processing the data in accordance with the program 122.

The input device 130 comprises, for example, a keyboard, a mouse, a touch panel, and a microphone. In addition, the input device 130 may comprise various sensors for measuring the patient status. Such sensors may be, for example, a wristwatch-type sensor and a vital sensor which are attached to the patient, and a camera for picking up the patient status, In this example embodiment, in a case where the input device 130 includes the touch panel and the output device 140 includes the display device, those devices may be integrally formed and configured as a display device with a touch panel.

The communication interface 150 is an interface for connecting the patient status determination device 100 and the totalization device 200, which will later be described, via a wire, wirelessly, or the like. The communication interface 150 is implemented, for example, by a transmitter, a transceiver, or a connector. In a case where the communication interface 150 comprises the transmitter, the transmitter modulates a carrier using a processed result (specifically, a determined score which will later be described) in the data processing device 110 and transmits a modulated wave as a transmitted wave to the totalization device 200 wirelessly. In a case where the communication interface 150 comprises the connector, a recording medium which is not shown in the figure may be used. In this event, the processed result (determined score) is written in the recording medium via the connector as recorded information with the patient ID added thereto.

In addition, the medical staff person may sometimes be required to perform a measure on the patient in question on measuring (determining) the patient status. Accordingly, it is desirable to use an earphone with a microphone or a headphone with a microphone as a combination of the above-mentioned receiving set and the above-mentioned microphone. Alternatively, the patient status determination device 100 may be kept on a transport table (bed) which carries the patient.

In this example embodiment, both of speeches and images (text data) are used as indications for the medical staff person and reactions of the medical staff person. Accordingly, the earphone with the microphone and the display device with the touch panel are provided as a combination of the input device 130 and the output device 140. However, in this invention, only the images (the text data) may be used as the indications for the medical staff person and the reactions of the medical staff person.

The storage device 120 comprises an item storage unit 124. In the item storage unit 124, a plurality of items required to measure the patient status within the above-mentioned measurement time interval (determination time interval) are preliminarily stored in a form of a plurality of questions associated with respective items in accordance with an order. In this example embodiment, the item storage unit 124 stores the plurality of questions as speech data and text data therein. For the plurality of items, allowed time intervals are preliminarily set, respectively, These allowed time intervals may be uniform or may be different from one another. In this example embodiment, the allowed time intervals are uniform. The allowed time intervals are stored in the item storage unit 124 in association with every item.

For example, such speech data and text data may be read out of a predetermined database and necessary information only may be preliminarily stored in the item storage unit 124 just before a starting time instant of the above-mentioned measurement time interval (determination time interval). Instead, in a case where the patient status determination device 100 is kept on the transport table (bed) of the patient, the following may be performed. First, from a control center which is not shown in the figure, a plurality of necessary items (speech data and text data) for the patient are read out of the predetermined database just before the starting time instant of the above-mentioned measurement time interval (determination time interval) and transmitted to the patient status determination device 100. The patient status determination device 100 may comprise a receiver (not shown) for receiving the transmitted necessary items and store the received necessary items in the item storage unit 124. In this example, the plurality of necessary items are preliminarily determined because those items are thirteen items defined by the above-mentioned NIHSS. Accordingly, instead, in the patient status determination device 100, the plurality of necessary items received by the receiver may be directly supplied to an indication unit 111 (which will later be described) of the data processing device 110 without using the item storage unit 124.

The data processing device 110 comprises, as main processing units, the indication unit 111, a reaction recognition unit 112, a score determination unit 113, a time measurement unit 114, and a control unit 118.

The indication unit 111 sequentially indicates, to the medical staff person, the plurality of items to be measured for the patient. The reaction recognition unit 112 recognizes a reaction from the medical staff person to the indicated item to produce a recognized result. The score determination unit 113 determines, based on the recognized result, a score for the indicated item to produce the determined score. The indication unit 111 presents, to the medical staff person, each of the plurality of items in the form of at least one question associated with the item. In this event, the indication unit 111 may present, to the medical staff person, the above-mentioned allowed time interval for every item. The time measurement unit 114 measures an elapsed time interval from the starting time instant when indication of each item to the medical staff person is started. The control unit 118 controls operation of the data processing device 110 as a whole. Hereinafter, operations of the respective processing units will be described more in detail.

The indication unit 111 sends the speech data read out of the item storage unit 124 to the earphone with the microphone of the output device 140. An earphone of the earphone with the microphone converts the speech data into a question speech. Accordingly, a combination of the indication unit 111 and the earphone of the output device 140 serves as a speech notification means for sequentially notifying the medical staff person of the plurality of items with the question speeches. In addition, a combination of the item storage unit 124, the indication unit 111, and the earphone of the output device 140 acts as a presentation unit for sequentially presenting, to the medical staff person, the plurality of necessary items required to measure the patient status with speeches.

Simultaneously, the indication unit 111 sends the text data read out of the item storage unit 124 to the display device with the touch panel of the output device 140. The display device with the touch panel displays the text data on a display screen thereof. Accordingly, a combination of the indication unit 111 and the display device with the touch panel of the output device 140 serves also as a text notification means for sequentially notifying the medical staff person of the plurality of items as question text data. Thus, a combination of the item storage unit 124, the indication unit 111, and the display device with the touch panel of the output device 140 acts as a presentation unit for sequentially presenting, to the medical staff person, the plurality of necessary items required to measure the patient status with the images (text data).

In response to the question speech generated from the earphone of the earphone with microphone of the output device 140, the medial staff person utters an answer (response) corresponding thereto towards the microphone of the earphone with the microphone of the input device 130. For example, it is assumed that “is there a reaction?” as the question speech flows from the earphone. In this event, the medical staff person answers (responds), for example, “yes” with a speech towards the microphone. The microphone converts an answer speech of “yes” into an input value of an answer speech signal which is an electric signal. Accordingly, the microphone of the earphone with the microphone serves as an input unit configured to input the answer speech uttered by the medical staff person in response to the indicated item, and to output the input value answer speech signal).

Simultaneously, in response to the question text data displayed on the display device with touch panel of the output device 140, the medical staff person operates the touch panel of the display device with touch panel as the input device 130 to input an answer (response) corresponding thereto. Herein, it is assumed that icons of “Yes” and “No” are displayed on the touch panel with touch panel. For example, it is assumed that the question text data displayed on the display device with touch panel is “it there a reaction?” In this event, the medical staff person answers (responds), for example, by touching the icon of “Yes” of the touch panel. The touch panel produces an answer text signal indicative of the answer of “Yes.” Accordingly, the touch panel of the display device with touch panel serves as an input unit configured to input the answer text signal by the medical staff person in response to the indicated item.

As described above, in this example embodiment, both of the speech and the image (text data) are used as the reaction from the medical staff person. However, in a noisy environment or the like, there is a case where only the image (text data) can be used as the reaction to the question. In this case, of course, only the image (text data) is used as the reaction from the medical staff person.

It is assumed that the input device 130 supplies the reaction recognition unit 112 with the answer speech signal uttered from the medical staff person as the reaction. The reaction recognition unit 112 includes a speech recognition unit (not shown). The speech recognition unit identifies the answer speech signal to produce an identified result as the above-mentioned recognized result. For instance, it is assumed that the answer speech signal is a speech signal indicative of “yes.” In this event, the speech recognition unit of the reaction recognition unit 112 produces a result of “Yes” as the recognized result (identified result).

On the other hand, it is assumed that the input device 130 supplies the reaction recognition unit 112 with the answer signal obtained by operation (touching) of the medical staff person as the reaction. In this event, the reaction recognition unit 112 recognizes the answer text signal to produce the recognized result. For instance, it is assumed that the answer text signal is a text signal indicative of “Yes,” In this event, the reaction recognition unit 112 produces a result of “Yes” as the recognized result.

The recognized result is supplied to the score determination unit 113. The score determination unit 113 determines, based on the recognized result, the score for the indicated item. For instance, it is assumed that the indicated item is “Level of Consciousness.” In this event, the indication unit 11 provides, to the medical staff person via the output device 140, an indication of “Is there a reaction?” together with an indication (guidance) of a measure for the patient. In response to the indication of “Is there a reaction?”, the score determination unit 113 is supplied from the reaction recognition unit 112 with the recognized result of “Yes” or “No.” The score determination unit 113 determines, based on the recognized result of “Yes” or “No”, the score (in this case, a score between 0 and 3) for the item of “Level of Consciousness.”

The totalization device 200 comprises a data processing device 210 for processing data, a storage device 220 for storing a program and data which will later be descried, and a communication interface 230. The data processing device 210 includes a totalization unit 212.

The communication interface 230 is an interface for connecting the totalization device 200 and the patient status determination device 100 described above via a wire, wirelessly, or the like. The communication interface 230 is implemented, for example, by a receiver, a transceiver, or a connector. In a case where the communication interface 230 comprises the receiver, the receiver receives, as a received wave, the transmitted wave which is transmitted from the communication interface (transmitter) 150 of the patient status determination device 100. The receiver demodulates the received wave to reproduce the above-mentioned processed result (determined score). In a case where the communication interface 230 comprises the connector, the recorded information (determined score and patient ID) recorded in the recording medium is read therefrom via the connector.

From the communication interface 230, the reproduced (read-out) determined score is supplied to the data processing device 210. The totalization unit 212 of the data processing device 210 totalizer the score. The totalization unit 212 stores a totalized result in the storage unit 220.

Now, effects of the first example embodiment will be described, As described above, the patient status determination device 100 according to the first example embodiment can appropriately determine the patient status by the medical staff person in a relatively short measurement time interval (determination time interval) without hesitation in scoring. This is because appropriate guidance and indication are provided for the medical staff person.

[Operation of Patient Status Determination Device]

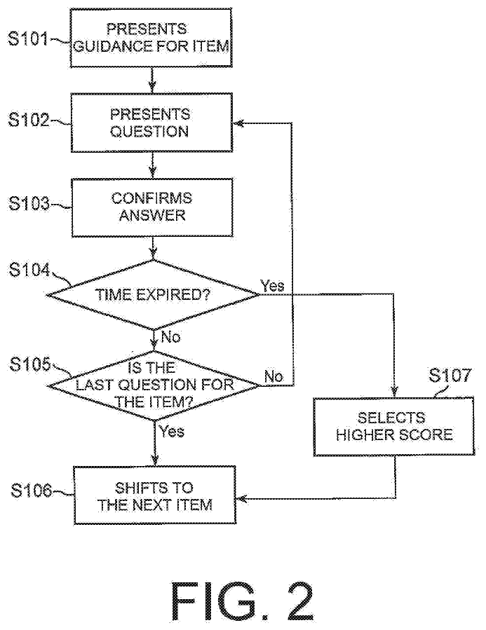

FIG. 2 is a flow chart for illustrating a flow of operation of the patient status determination device 100 illustrated in FIG. 1. Now, the flow of the operation of the patient status determination device 100 will be described with reference to FIGS. 1 and 2.

First, the indication unit 111 reads an item out of the item storage unit 124 to cause the receiving set and the display device of the output device 140 to present guidance of the item (Step S101). In this event, the indication unit 111 may cause the display device to present, on the display screen thereof, an allowed time interval set for the item. Simultaneously, the control unit 118 starts the time measurement unit 114 to cause the time measurement unit 114 to measure an elapsed time interval from a starting time instant when such indication is started.

Subsequently, the indication unit 111 uses the earphone and the display device of the output device 140 to present a question of the item as a question speech and question text data (Step S102).

In response to the question speech and the question text data, the medical staff person utters an answer speech from the microphone of the input device 130 or inputs an answer by operating the touch panel of the input device 130. The reaction recognition unit 112 recognizes (confirms) the answer speech signal or the answer text signal to produce a recognized result (confirmed result) (Step S103).

The control unit 118 determines whether or not the elapsed time interval measured by the time measurement unit 114 exceeds the above-mentioned allowed time interval (Step S104). When the elapsed time interval does not exceed the allowed time interval (No in Step S104), the control unit 118 determines whether or not the question is the last question in the item (Step S105). If it is not the last question (No in Step S105), the control unit 118 causes the indication unit 111 to present the next question (Step S102). If it is the last question (Yes in Step S105), the score determination unit 113 determines a score for the item based on the recognized result(s) up to then and the control unit 118 controls the indication unit 111 to shift to the next item (Step S106).

When the elapsed time interval measured by the time measurement unit 114 exceeds the allowed time interval (Yes in Step S014), the score determination unit 113 selects and produces, as a determined score, a higher score as a reaction to the question at that time instant (Step S107) and the control unit 118 controls the indication unit 111 to shift to the next item (Step S106).

### Second Example Embodiment

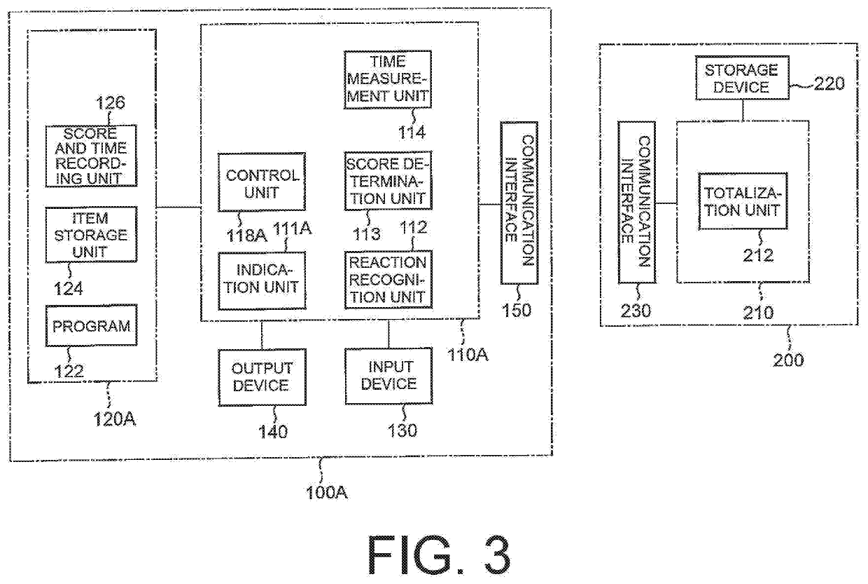

FIG. 3 is a block diagram for illustrating a configuration of a patient status determination system according to a second example embodiment of the present invention. As illustrated in FIG. 3, the patient status determination system comprises a patient status determination device 100A and the totalization device 200.

The patient status determination device 100A is similar in structure and operation to the patient status determination device 100 illustrated in FIG. 1 except that processed contents in the data processing device and stored contents of the storage device are different as will later be described. The data processing device and the storage device are therefore depicted by the reference numerals 110A and 120A, respectively. The same reference numerals are assigned to components similar in function to those illustrated in FIG. 1 and, hereinafter, differences alone will be described for the sake of simplification of the description.

The data processing device 110A carries out processing operations similar to those of the data processing device 110 illustrated in FIG. 1 except that an indication unit 111A and a control unit 118A are provided in place of the indication unit 111 and the control unit 118. On the other hand, the storage device 120A is similar in structure and operation to the storage device 120 illustrated in FIG. 1 except that a score and time recording unit 126 is further provided. That is, the storage device 120A comprises the program 122, the item storage unit 124, and the score and time recording unit 126.

The score and time recording unit 126 records, as recorded information, the determined score determined by the score determination unit 113 and the elapsed time interval at the time measurement unit 114 in association with the indicated item. In other words, the control unit 118A causes the score and time recording unit 126 to store, as the recorded information, the determined score determined by the score determination unit 113 and the elapsed time interval at the time measurement unit 114 in association with the indicated item. In addition, the staff ID (user ID) may also be recorded as the recorded information.

The indication unit 111A changes, based on the recorded information recorded in the score and time recording unit 126 during past measurements including at least the last time, notification contents to be indicated to the medical staff person in a current measurement. The changed notification contents include the allowed time interval which is set for the indicated item. That is, in the second example embodiment, the allowed time interval is adjusted for each item. In addition, as the changed notification contents, the display device with touch panel of the output device 140 may display the score given to the item in the past in combination with the question. In this event, the medical staff person can react with reference to the displayed contents also. As a result, it is possible to shorten the measurement time interval (determination time interval) and to carry out appropriate scoring.

Now, effects of the second example embodiment will be described. The patient status determination device 100A according to the second example embodiment can carry out appropriate determination with the measurement time interval (determination time interval) shortened as compared with the case of the above-mentioned first example embodiment. This is because the indication unit 111A carries out appropriate guidance and indication for the medical staff person by feeding back the measured results in the past.

### Third Example Embodiment

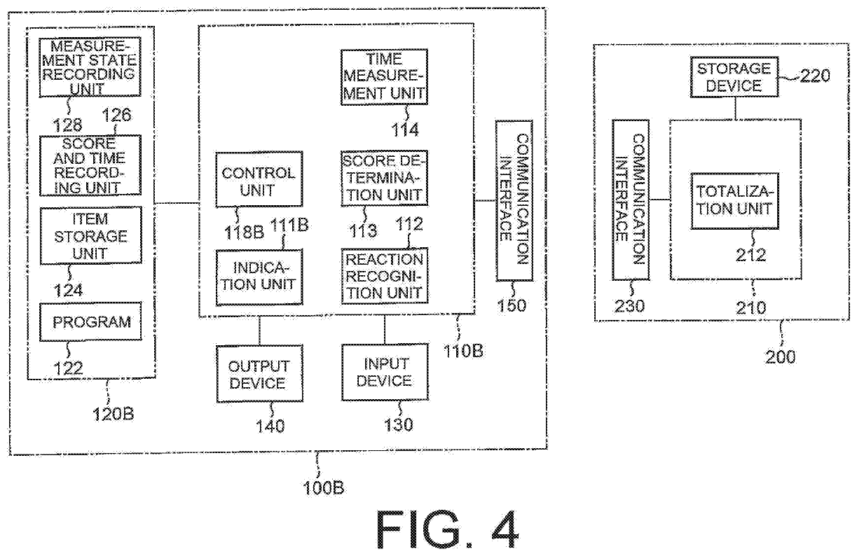

FIG. 4 is a block diagram for illustrating a configuration of a patient status determination system according to a third example embodiment of the present invention. As illustrated in FIG. 4, the patient status determination system comprises a patient status determination device 100B and the totalization device 200.

The patient status determination device 100B is similar in structure and operation to the patient status determination device 100A illustrated in FIG. 3 except that processed contents in the data processing device and stored contents of the storage device are different as will later be described. The data processing device and the storage device are therefore depicted by the reference numerals 110B and 120B, respectively. The same reference numerals are assigned to components similar in function to those illustrated in FIG. 3 and, hereinafter, differences alone will be described for the sake of simplification of the description.

The data processing device 110B carries out processing operations similar to those of the data processing device 110A illustrated in FIG. 3 except that an indication unit 111B and a control unit 118B are provided in place of the indication unit 111A and the control unit 118A. On the other hand, the storage device 120B is similar in structure and operation to the storage device 120A illustrated in FIG. 3 except that a measurement state recording unit 128 is further provided. That is, the storage device 120B comprises the program 122, the item storage unit 124, the score and time recording unit 126, and the measurement state recording unit 128.

Although not-illustrated in the figure, as described above, the wristwatch-type sensor and the vital sensor are attached to the patient and the camera for picking up the patient is equipped in the transport table (bed) for transporting the patient or in a sick room for accommodating the patient. The measurement state recording unit 128 records, as a measurement state, data acquired from those sensors and an image picked-up by the camera. In other words, the control unit 118B causes the measurement state recording unit 128 to store, as the measurement state, the vital data acquired from those sensors and the image picked-up by the camera.

The indication unit 111B changes, based on the recorded information recorded in the score and time recording unit 126 and the measurement state recorded in the measurement state recording unit 128 during past measurements including at least the last time, notification contents to be indicated to the medical staff person in a current measurement. The changed notification contents include the allowed time interval which is set for the indicated item, like in the above-mentioned second example embodiment. That is, in the third example embodiment also, the allowed time interval is adjusted for each item. In addition, as the changed notification contents, the display device with touch panel of the output device 140 may display the score given to the item in the past, the vital data, and the image of the patient in combination with the question. In this event, the medical staff person can react with reference to these displayed contents also. As a result, it is possible to furthermore shorten the measurement time interval (determination time interval) and to carry out more appropriate scoring.

Now, effects of the third example embodiment will be described, The patient status determination device 100B according to the third example embodiment can carry out more appropriate determination with the measurement time interval (determination time interval) furthermore shortened as compared with the case of the above-mentioned second example embodiment. This is because the indication unit 111B carries out appropriate guidance and indication for the medical staff person by feeding back the measured results in the past and the condition of the patient.

[Operation of Patient Status Determination Device]

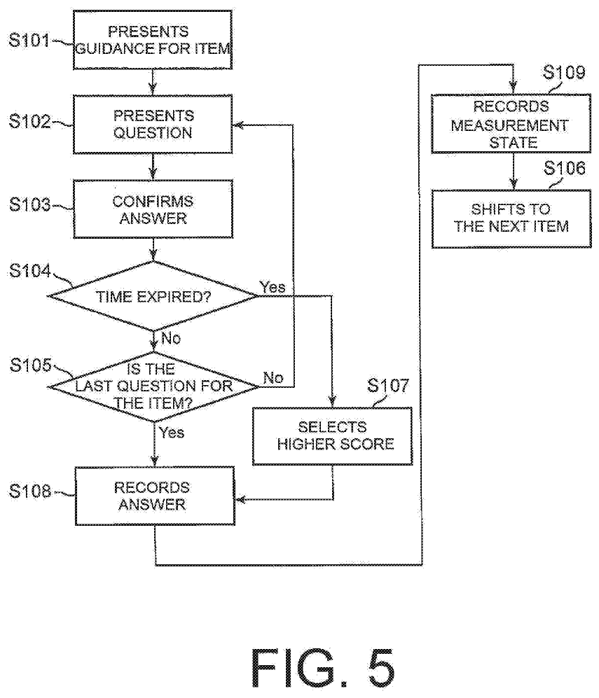

FIG. 5 is a flow chart for illustrating a flow of operation of the patient status determination device 100B illustrated in FIG. 4. Now, the flow of the operation of the patient status determination device 100B will be described with reference to FIGS. 4 and 5.

Operations in Steps S101 to S106 of FIG. 5 are similar to those in the Steps S101 to S106 of FIG. 2 and therefore the description thereof is omitted.

It is assumed that determination of a score by the score determination unit 112 finishes after the last question (Yes in Step S105) or that selection of the score by the score determination unit 112 (Step S107) finishes after the elapsed time interval at the time measurement unit 114 exceeds the allowed time interval (Yes in Step S104). Thereafter, the control unit 118B causes the score and time recording unit 126 to store, as the recorded information, the score determined by the score determination unit 113 and the elapsed time interval at the time measurement unit 114 in association with the indicated item (Step S108).

Subsequently, the control unit 118B causes the measurement state recording unit 128 to store, as the measurement state, the vital data acquired from the above-mentioned sensors and the image picked-up by the camera (Step S109). Thereafter, the control unit 118B controls the indication unit 11B to shift to the next item (Step S106).

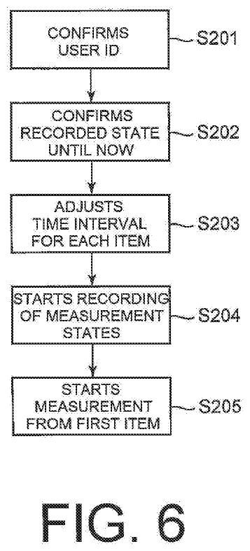

Now, referring to FIG. 6, description will proceed to an operation of changing the notification contents to be indicated by the indication unit 111B by giving feedback before starting a measurement.

First, the control unit 118B confirms the user ID (staff ID) (Step S201). Subsequently, the control unit 118B reads the recorded information and the measurement state out of the score and time recording unit 126 and the measurement state recording unit 128, respectively, to confirm a recorded state of the patient until now (Step S202). The control unit 118B adjusts, based on the recorded state, the allowed time interval which is set for each item (Step S203). Thereafter, the control unit 118B starts the above-mentioned sensors and the cameral to start recording of measurement states (Step S204). Then, the control unit 118B controls the indication unit 111B to start the measurement from the first item (Step S205).

### Fourth Example Embodiment

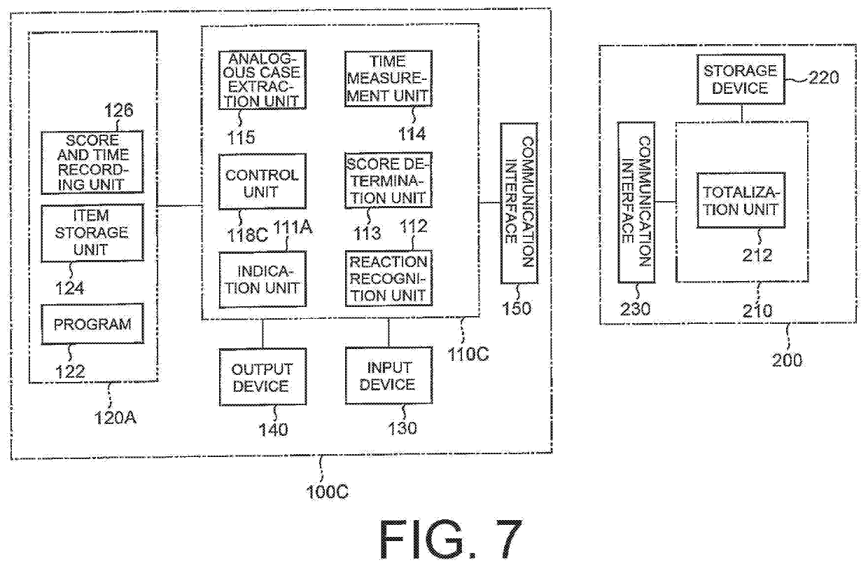

FIG. 7 is a block diagram for illustrating a configuration of a patient status determination system according to a fourth example embodiment of the present invention. As illustrated in FIG. 7, the patient status determination system comprises a patient status determination device 100C and the totalization device 200.

The patient status determination device 100C is similar in structure and operation to the patient status determination device 100A illustrated in FIG. 3 except that processed contents in the data processing device are different as will later be described. The data processing device is therefore depicted by the reference numeral 110C. The same reference numerals are assigned to components similar in function to those illustrated in FIG. 3 and, hereinafter, differences alone will be described for the sake of simplification of the description.

The data processing device 110C carries out processing operations similar to those of the data processing device 110A illustrated in FIG. 3 except that a control unit 118B is provided in place of the control unit 118A and that an analogous case extraction unit 115 is further provided.

The patient status determination device 100C is communicably connected to an analogous case database which is not shown in the figure. The analogous case database is a database which preliminarily saves a plurality of analogous cases.

In a case where the reaction cannot be obtained from the medical staff person at a time instant when the elapsed time interval measured by the time measurement unit 114 exceeds the above-mentioned allowed time interval, the analogous case extraction unit 115 extracts, from the analogous case database, an analogous case which is analogous to a status of the patient and presents the extracted analogous case to the medical staff person.

Now, effects of the fourth example embodiment will be described. The patient status determination device 100C according to the fourth example embodiment can carry out more appropriate determination, as compared with the above-mentioned second example embodiment. This is because the analogous case in the past is presented to the medical staff person.

[Operation of Patient Status Determination Device]

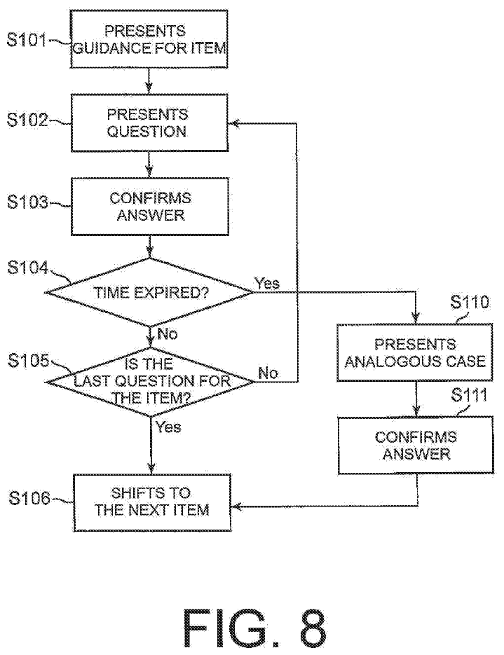

FIG. 8 is a flow chart for illustrating a flow of operation of the patient status determination device 100C illustrated in FIG. 7. Now, the flow of the operation of the patient status determination device 100C will be described with reference to FIGS. 7 and 8.

Operations in Steps S101 to S106 of FIG. 8 are similar to those in Steps S101 to S106 of FIG. 2 and therefore the description thereof is omitted. FIG. 8 is different from FIG. 2 in that Steps S110 and S111 are provided instead of the Step S107 in FIG. 2.

When the elapsed time interval measured by the time measurement unit 114 exceeds the allowed time interval (Yes in Step S104), the analogous case extraction unit 115 extracts, from the analogous case database, an analogous case which is analogous to a status of the patient in question and presents the extracted analogous case by displaying it on the display device with touch panel of the output device 140 (Step S110). Accordingly, the medical staff person can react with reference to the displayed analogous case. The reaction recognition unit 112 recognizes the reaction from the medical staff person to produce a recognized result (Step S111). The score determination unit 113 determines a score for the specified item based on the recognized result and the control unit 118C controls the indication unit 111A to shift to the next item (Step S106).

### EXAMPLE

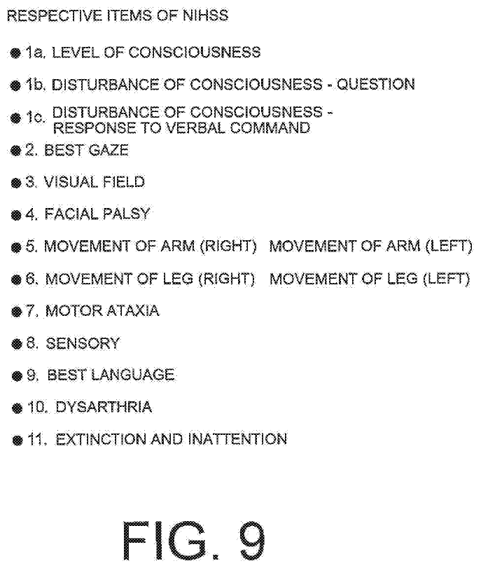

Now, referring to FIGS. 9 to 22, description will proceed to a patient status determination device according to an example of the present invention. In the illustrated example, for convenience of explanation, description will be made about a case where it is applied to the above-mentioned patient status determination device 100 according to the first example embodiment illustrated in FIGS. 1 and 2. As a matter of course, this example is similarly applicable to the patient status determination devices 100A to 100C according to the other example embodiments.

FIG. 9 is a view for illustrating thirteen items of the NIHSS. These thirteen items are well known in this technical field and, therefore, are not described in detail. Hereinafter, referring to FIGS. 1 and 2, description will proceed to the operation of determining, for each of the thirteen items of the NIHSS, the patient status using the patient status determination device 100 illustrated in FIG. 1. As described above, for these thirteen items, the guidance and the speech data and the text data of the questions are preliminarily stored in the item storage unit 124. In the figures described in the following, G indicates the guidance. As described above, the guidance and the questions are carried out by both of speeches and images (text data).

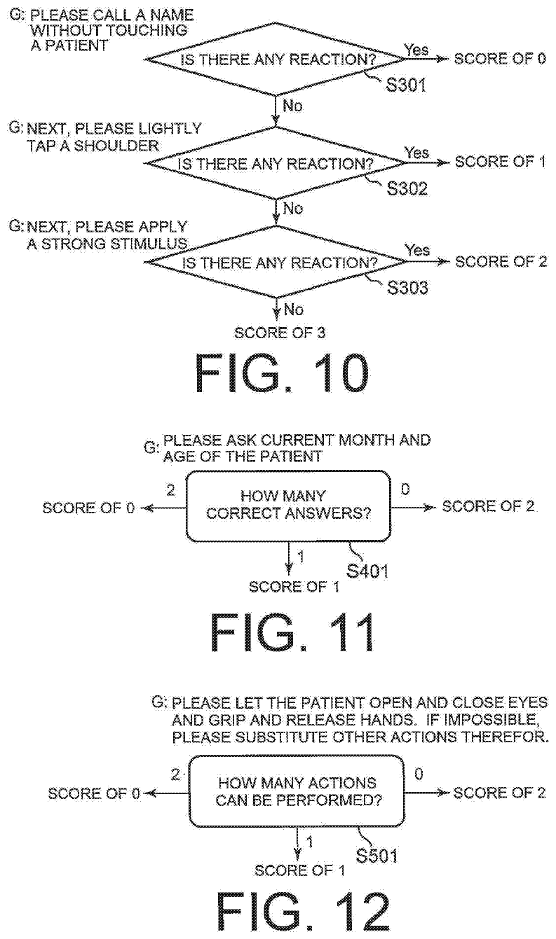

FIG. 10 is a flow chart for illustrating guidance and questions which are indicated by the indication unit 111 for an item of “1a. Level of Consciousness.”

First, in the Step S101 in FIG. 2, the indication unit 111 use the output device 140 to present the guidance of “Please call a name without touching a patient.” Subsequently, in the Step S102 in FIG. 2, the indication unit 111 uses the output device 140 to present a question of “Is there any reaction?” (Step S301). Herein, if there is any reaction of the patient, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S301) to produce a recognized result thereof.

In this event, this question is the last question for this item (Yes in Step S105 of FIG. 2). Then, based on the recognized result, the score determination unit 113 determines a score for the item of “Level of Consciousness” as zero. Thereafter, the processing shifts to the next item (see Step S106 in FIG. 2).

On the other hand, if there is no reaction of the patient in the above-mentioned Step S301, the medical staff person answers “no” with a speech from the microphone or touches the icon of “No” on the display screen of the display device with touch panel.

In this event, since this question is not the last question for this item (No in Step S105 of FIG. 2), the indication unit 111 presents the guidance of “Next, please lightly tap a shoulder.” (Step S101 in FIG. 2) and then presents a question of “Is there any reaction?” (Step S102 in FIG. 2 and Step S302 in FIG. 10). Herein, if there is any reaction of the patient, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S302) to produce a recognized result thereof.

In this event, since this question is the last question for this item (Yes in Step S105 of FIG. 2), the score determination unit 113 determines, based on the recognized result, a score for the item of “Level of Consciousness” as one. Thereafter, the processing shifts to the next item (see Step S106 in FIG. 2).

On the other hand, if there is no reaction of the patient in the above-mentioned Step S302, the medical staff person answers “no” with a speech from the microphone or touches the icon of “No” on the display screen of the display device with touch panel.

In this event, since this question is not the last question for this item (No in Step S105 of FIG. 2), the indication unit 111 presents the guidance of “Next, please apply a strong stimulus.” (Step S101 in FIG. 2) and then presents a question of “Is there any reaction?” (Step S102 in FIG. 2 and Step S303 in FIG. 10). Herein, if there is any reaction of the patient, the medical staff person answers “yes” with a speech front the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S303) to produce a recognized result thereof.

In this event, since this question is the last question for this item (Yes in Step S105 of FIG. 2), the score determination unit 113 determines, based on the recognized result, a score for the item of “Level of Consciousness” as two. Thereafter, the processing shifts to the next item (see Step S106 in FIG. 2).

On the other hand, if there is no reaction of the patient in the above-mentioned Step S302, the medical staff person answers “no” with a speech from the microphone or touches the icon of “No” on the display screen of the display device with touch panel. In this event, since this question is the last question for this item (Yes in Step S105 of FIG. 2), the score determination unit 113 determines, based on the recognized result, a score for the item of “Level of Consciousness” as three. Thereafter, the processing shifts to the next item (see Step S106 in FIG. 2).

On the other hand, in the above-mentioned Step S303, it is assumed that the medical staff person hesitates in determination, so that the elapsed time interval exceeds the allowed time interval (Yes in Step S104 in FIG. 2). In this event, the score determination unit 113 selects and produces, as a determined score, a higher score as a reaction of the medical staff person to the question at this time instant (Step 107 in FIG. 2). In this example, since the reaction of “No” has a score higher than that of the reaction of “Yes”, the score determination unit 113 selects and produces three as a score for the item of “Level of Consciousness.”

FIG. 11 is a flow chart for illustrating guidance and a question which are indicated by the indication unit 111 for an item of “1b. Disturbance of Consciousness—Question.”

First, in the Step S101 in FIG. 2, the indication unit Iii uses the output device 140 to present the guidance of “Please ask the current month and the age of the patient.” Subsequently, in the Step S102 in FIG. 2, the indication unit 111 uses the output device 140 to present the question of “How many correct answers?” (Step S401). Herein, if the number of the correct answer of the patient is zero, the medical staff person answers “zero” with a speech from the microphone or touches a key button of “0” on the display screen of the display device with touch panel. As a result, in the Step S103 in FIG. 2, the reaction recognition unit 112 recognizes the speech of “zero” or touching of “0” (“0” in Step S401) to produce a recognized result thereof.

In this event, since this question is the last question for this item (Yes in Step S105 in FIG. 2), the score determination unit 113 determines, based on the recognized result, a score for the item of “Disturbance of Consciousness—Question” as two. Thereafter, the processing shifts to the next item (see Step S106 in FIG. 2).

In the Step S401, if the number of the correct answer of the patient is one, the medical staff person answers “one” with a speech from the microphone or touches a key button of “1” on the display screen of the display device with touch panel. As a result, in the Step S103 in FIG. 2, the reaction recognition unit 112 recognizes the speech of “one” or touching of “1” (“1” in Step S401) to produce a recognized result thereof.

In this event, since this question is the last question for this item (Yes in Step S105 in FIG. 2), the score determination unit 113 determines, based on the recognized result, a score for the item of “Disturbance of Consciousness—Question” as one. Thereafter, the processing shifts to the next item (see Step S106 in FIG. 2).

In the Step S401, if the number of the correct answers of the patient is two, the medical staff person answers “two” with a speech from the microphone or touches a key button of “2” on the display screen of the display device with touch panel. As a result, in the Step S103 in FIG. 2, the reaction recognition unit 112 recognizes the speech of “two” or touching of “2” (“2” in Step S401) to produce a recognized result thereof.

In this event, since this question is the last question for this item (Yes in Step S105 in FIG. 2), the score determination unit 113 determines, based on the recognized result, a score for the item of “Disturbance of Consciousness Question” as zero. Thereafter, the processing shifts to the next item (see Step S106 in FIG. 2).

On the other hand, in the above-mentioned Step S401, it is assumed that the medical staff person hesitates in determination, so that the elapsed time interval exceeds the allowed time interval (Yes in Step S104 in FIG. 2). In this event, the score determination unit 113 selects and produces, as the determined score, a higher score as a reaction of the medical staff person to the question at this time instant (Step 107 in FIG. 2). In this example, since the reaction of “0” has a score higher than those of the reactions of “1” and “2”, the score determination unit 113 selects and produces two as a score for the item of “Disturbance of Consciousness Question.”

FIG. 12 is a flow chart for illustrating guidance and a question which are indicated by the indication unit 111 for an item of “1c. Disturbance of Consciousness—Response to Verbal Command.”

First, in the Step S101 in FIG. 2, the indication unit 111 uses the output device 140 to present the guidance of “Please let the patient open and close eyes and grip and release hands. If impossible, please substitute other actions therefor.” Subsequently, in the Step S102 in FIG. 2, the indication unit 11 uses the output device 140 to present the question of “How many actions can be performed?” (Step S501). Herein, if the number of the patient's actions is zero, the medical staff person answers “zero” with a speech from the microphone or touches the key button of “0” on the display screen of the display device with touch panel. As a result, in the Step S103 in FIG. 2, the reaction recognition unit 112 recognizes the speech of “zero” or touching of “0” (“0” in Step S401) to produce a recognized result thereof.

In this event, since this question is the last question for this item (Yes in Step S105 in FIG. 2), the score determination unit 113 determines, based on the recognized result, a score for the item of “Disturbance of Consciousness t Response to Verbal Command” as two. Thereafter, the processing shifts to the next item (see Step S106 in FIG. 2).

In the Step S501, if the number of the patient's actions is one, the medical staff person answers “one” with a speech from the microphone or touches the key button of “1” on the display screen of the display device with touch panel. As a result, in the Step S103 in FIG. 2, the reaction recognition unit 112 recognizes the speech of “one” or touching of “1” (“1” in Step S401) to produce a recognized result thereof.

In this event, since this question is the last question for this item (Yes in Step S105 in FIG. 2), the score determination unit 113 determines, based on the recognized result, a score for the item of “Disturbance of Consciousness—Response to Verbal Command” as one. Thereafter, the processing shifts to the next item (see Step S106 in FIG. 2).

In the Step S501, if the number of the patient's actions is two, the medical staff person answers “two” with a speech from the microphone or touches the key button of “2” on the display screen of the display device with touch panel. As a result, in the Step S103 in FIG. 2, the reaction recognition unit 112 recognizes the speech of “two” or touching of “2” (“2” in Step S401) to produce a recognized result thereof.

In this event, since this question is the last question for this item (Yes in Step S105 in FIG. 2), the score determination unit 113 determines, based on the recognized result, a score for the item of “Disturbance of Consciousness—Response to Verbal Command” as zero. Thereafter, the processing shifts to the next item (see Step S106 in FIG. 2).

On the other hand, in the above-mentioned Step S501, it is assumed that the medical staff person hesitates in determination, so that the elapsed time interval exceeds the allowed time interval (Yes in Step S104 in FIG. 2). In this event, the score determination unit 113 selects and produces, as the determined score, a higher score as a reaction of the medical staff person to the question at this time instant (Step 107 in FIG. 2). In this example, since the reaction of “0” has a score higher than those of the reactions of “1” and “2”, the score determination unit 113 selects and produces two as a score for the item of “Disturbance of Consciousness—Response to Verbal Command.”

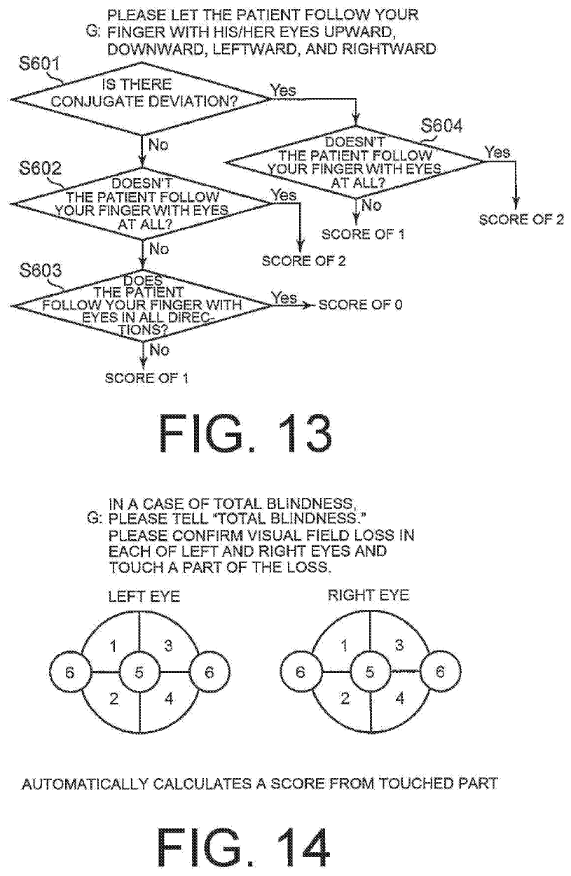

FIG. 13 is a flow chart for illustrating guidance and questions which are indicated by the indication unit Iii for an item of “2. Best Gaze.”

First, in the Step S101 in FIG. 2, the indication unit 111 uses the output device 140 to present the guidance of “Please let the patient follow your finger with his/her eyes upward, downward, leftward, and rightward.” Subsequently, in the Step S102 in FIG. 2, the indication unit 111 uses the output device 140 to present a question of “Is there conjugate deviation?” (Step S601).

Herein, it is assumed that the medical staff person judges that “there is no conjugate deviation” (No in Step S601). In this event, the medical staff person answers “no” with a speech from the microphone or touches the icon of “No” on the display screen of the display device with touch panel. As a result, in the Step S103 in FIG. 2, the reaction recognition unit 112 recognizes the speech of “no” or touching of “No” (No in Step S601) to produce a recognized result thereof.

In this case, since this question is not the last question for this item (No in Step S105 of FIG. 2), the indication unit 111 returns to the Step S102 of FIG. 2 and then uses the output device 140 to present a question of “Doesn't the patient follow your finger with his/her eves at all?” (Step S602).

Herein, it is assumed that the medical staff person judges that “the patient does not follow your finger with his/her eyes at all” (Yes in Step S602). In this event, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S602) to produce a recognized result thereof.

In this event, since this question is the last question for this item (Yes in Step S105 in FIG. 2), the score determination unit 113 determines, based on the recognized result, a score for the item of “Best Gaze” as two. Thereafter, the processing shifts to the next item (see Step S106 in FIG. 2).

On the other hand, in the above-mentioned Step S602, it is assumed that the medical staff person judges that “the patient follows your finger with his/her eyes” (No in Step S602). In this event, the medical staff person answers “no” with a speech from the microphone or touches the icon of “No” on the display screen of the display device with touch panel. As a result, in the Step S103 in FIG. 2, the reaction recognition unit 112 recognizes the speech of “no” or touching of “No” (No in Step S602) to produce a recognized result thereof.

In this case, since this question is not the last question for this item (No in Step S105 of FIG. 2), the indication unit 111 returns to the Step S102 in FIG. 2 and then uses the output device 140 to present a question of “Does the patient follow your finger with his/her eyes in all directions?” (Step S603).

Herein, it is assumed that the medical staff person judges that “the patient completely follows your finger with his/her eyes in all directions” (Yes in Step S603). In this event, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S603) to produce a recognized result thereof.

In this event, since this question is the last question for this item (Yes in Step S105 in FIG. 2), the score determination unit 113 determines, based on the recognized result, a score for the item of “Best Gaze” as zero. Thereafter, the processing shifts to the next item (see Step S106 in FIG. 2).

On the other hand, in the above-mentioned Step S603, it is assumed that the medical staff person judges that “the patient does not follow your finger with his/her eyes in all directions” (No in Step S603). In this event, the medical staff person answers “no” with a speech from the microphone or touches the icon of “No” on the display screen of the display device with touch panel. As a result, in the Step S103 in FIG. 2, the reaction recognition unit 112 recognizes the speech of “no” or touching of “No” (YNo in Step S603) to produce a recognized result thereof

In this event, since this question is the last question for this item (Yes in Step S105 in FIG. 2), the score determination unit 113 determines, based on the recognized result, a score for the item of “Best Gaze” as one. Thereafter, the processing shifts to the next item (see Step S106 in FIG. 2).

In the above-mentioned Step S601, it is assumed that the medical staff person judges that “there is conjugate deviation” (Yes in Step S601). In this event, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S601) to produce a recognized result thereof.

In this case, since this question is not the last question for this item (No in Step S105 of FIG. 2), the indication unit 111 returns to the Step S102 of FIG. 2 and then uses the output device 140 to present a question of “Doesn't the patient follow your finger with his/her eyes at all?” (Step S604).

Herein, it is assumed that the medical staff person judges that “the patient does not follow your finger with his/her eyes at all” (Yes in Step S604). In this event, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S604) to produce a recognized result thereof.

In this event, since this question is the last question for this item (Yes in Step S105 in FIG. 2), the score determination unit 113 determines, based on the recognized result, a score for the item of “Best Gaze” as two. Thereafter, the processing shifts to the next item (see Step S106 in FIG. 2).

On the other hand, in the above-mentioned Step S604, it is assumed that the medical staff person judges that “the patient follow your finger with his/her eyes” (No in Step S604). In this event, the medical staff person answers “no” with a speech from the microphone or touches the icon of “No” on the display screen of the display device with touch panel. As a result, in the Step S103 in FIG. 2, the reaction recognition unit 112 recognizes the speech of “yes” or touching of “Yes” (No in Step S604) to produce a recognized result thereof.

In this event, since this question is the last question for this item (Yes in Step S105 in FIG. 2), the score determination unit 113 determines, based on the recognized result, a score for the item of “Best Gaze” as one. Thereafter, the processing shifts to the next item (see Step S106 in FIG. 2).

On the other hand, in the above-mentioned Step S603, it is assumed that the medical staff person hesitates in determination, so that the elapsed time interval exceeds the allowed time interval (Yes in Step S104 in FIG. 2). In this event, the score determination unit 113 selects and produces, as the determined score, a higher score as a reaction of the medical staff person to the question at this time instant (Step 107 in FIG. 2). In this example, since the reaction of “No” has a score higher than that of the reaction of “Yes”, the score determination unit 113 selects and produces one as a score for the item of “Best Gaze.”

In addition, in the above-mentioned Step S604, it is assumed that the medical staff person hesitate in determination, so that the elapsed time interval exceeds the allowed time interval (Yes in Step S104 in FIG. 2). In this event, the score determination unit 113 selects and produces, as the determined score, a higher score as a reaction of the medical staff person to the question at this time instant (Step 107 in FIG. 2). In this example, since the reaction of “Yes” has a score higher than that of the reaction of “No”, the score determination unit 113 selects and produces two as a score for the item of “Best Gaze.”

FIG. 14 is a flow chart for illustrating guidance and a question which are indicated by the indication unit 111 for an item of “3. Visual Field” and an example of display on the display screen of the display device with touch panel.

First, in the Steps S101 and S102 in FIG. 2, the indication unit 111 uses the output device 140 to present the guidance and the question of “Please tell “total blindness” in a case of total blindness.” Please confirm visual field loss in each of left and right eyes of the patient and touch a part of the loss.” In a case of this item, as shown in FIG. 14, an image in which the numbers of 1 to 6 are written for the left eye and the right eve is displayed on the display screen of the display device with touch panel.

The medical staff person observes the patient and touches the part of the loss on the displayed image. The reaction recognition unit 112 produces a touch signal corresponding to a touched part as the recognized result (Step S103 in FIG. 2). When a touch operation of the medical staff person finishes, this touch operation corresponds to the last question for this item (Yes in Step S105 of FIG. 2). Therefore, the score determination unit 113 automatically calculates and produces a score based on the touch signal (recognized result).

On the other hand, it is assumed that the medical staff person hesitates in determination, so that the elapsed time interval exceeds the allowed time interval (Yes in Step S104 in FIG. 2). In this event, the score determination unit 113 selects and produces, as the determined score, a highest score as a reaction of the medical staff person to the question at this time instant (Step 107 in FIG. 2).

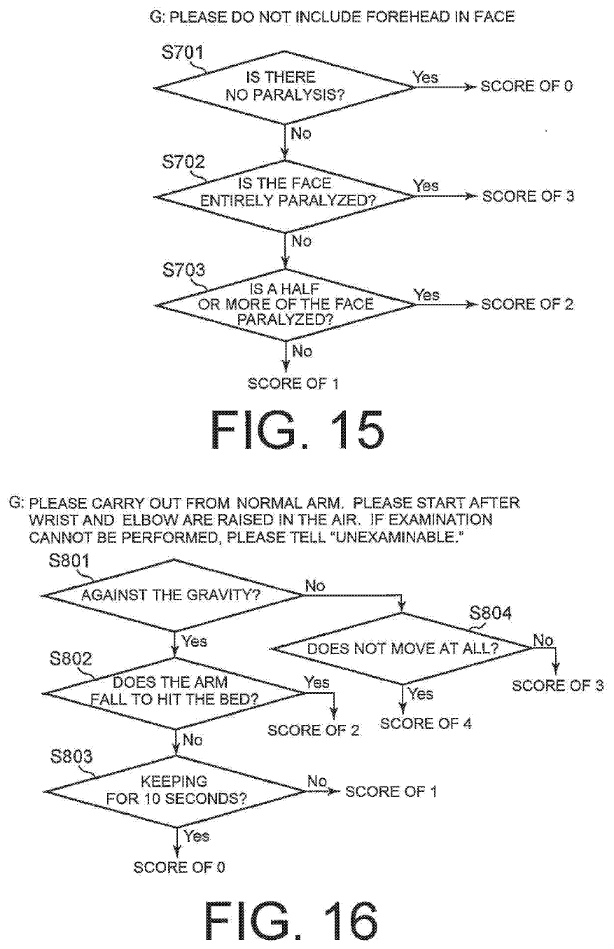

FIG. 15 is a flow chart for illustrating guidance and questions which are indicated by the indication unit 111 for an item of “4. Facial Palsy.”

First, in the Step S101 in FIG. 2, the indication unit 111 uses the output device 140 to present the guidance of “Please do not include a forehead in a face.” Subsequently, in the Step S102 in FIG. 2, the indication unit 111 uses the output device 140 to present the question of “Is there no paralysis?” (Step S701). Herein, if the medical staff person judges that there is no paralysis in the face of the patient, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S701) to produce a recognized result thereof.

In this event, since this question is the last question for this item (Yes in Step S105 in FIG. 2), the score determination unit 113 determines, based on the recognized result, a score for the item of “Facial Palsy” as zero. Thereafter, the processing shifts to the next item (see Step S106 in FIG. 2).

On the other hand, in the above-mentioned Step S701, if the medical staff person judges that there is any paralysis in the face of the patient, the medical staff person answers “no” with a speech from the microphone or touches “No” on the display screen of the display device with touch panel.

In this case, since this question is not the last question for this item (No in Step S105 of FIG. 2), the indication unit 111 presents a question of “Is the face of the patient entirely paralyzed?” (Step S102 in FIG. 2 and Step S702 in FIG. 15). Herein, if the medical staff person judges that the face of the patient is entirely paralyzed, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S702) to produce a recognized result thereof.

In this event, since this question is the last question for this item (Yes in Step S105 in FIG. 2), the score determination unit 113 determines, based on the recognized result, a score for the item of “Facial Palsy” as three. Thereafter, the processing shifts to the next item (see Step S106 in FIG. 2).

On the other hand, in the above-mentioned Step S702, if the medical staff person judges that the face of the patient is not entirely paralyzed, the medical staff person answers “no” with a speech from the microphone or touches “No” on the display screen of the display device with touch panel.

In this case, since this question is not the last question for this item (No in Step S105 of FIG. 2), the indication unit 111 presents a question of “Is a half or more of the face of the patient paralyzed?” (Step S102 in FIG. 2 and Step S703 in FIG. 15). Herein, if the medical staff person judges that the half or more of the face of the patient is paralyzed, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S702) to produce a recognized result thereof.

In this event, since this question is the last question for this item (Yes in Step S105 in FIG. 2), the score determination unit 113 determines, based on the recognized result, a score for the item of “Facial Palsy” as two. Thereafter, the processing shifts to the next item (see Step S106 in FIG. 2).

On the other hand, in the above-mentioned Step S703, if the medical staff person judges that the half or more of the face of the patient is not paralyzed, the medical staff person answers “no” with a speech from the microphone or touches the icon of “No” on the display screen of the display device with touch panel. In this case, since this question is the last question for this item (No in Step S105 of FIG. 2), the score determination unit 113 determines, based on the recognized result, a score for the item of “Facial Palsy” as one. Thereafter, the processing shifts to the next item (see Step S106 in FIG. 2).

On the other hand, in the above-mentioned Step S703, it is assumed that the medical staff person hesitates in determination, so that the elapsed time interval exceeds the allowed time interval (Yes in Step S104 in FIG. 2). In this event, the score determination unit 113 selects and produces, as the determined score, a higher score as a reaction of the medical staff person to the question at this time instant (Step 107 in FIG. 2). In this example, since the reaction of “Yes” has a score higher than that of the reaction of “No”, the score determination unit 113 selects and produces two as a score for the item of “Facial Palsy.”

Like in this example, in a case where the item is a specific item such as “Facial Palsy”, the indication unit 111 first indicates, as a first one of a plurality of questions for the specific item, a question for which the medical staff person can easily judge the status of the patient (in this example, “Is there no paralysis?”). Then, the indication unit 111 indicates, as a last one of the plurality of questions for the specific item, a question for which the medical staff person hardly judge the status of the patient (in this example, “Is a half or more of the face of the patient paralyzed?”).

By selecting the order of the questions to be presented in this manner, more accurate scoring is possible in this example. In detail, if the questions are not made in such an order, the medical staff person observes the face of the patient and must judge, by him/herself, whether or not there is any paralysis and, in presence of the paralysis, the level of the paralysis. Such judgment is very difficult and, therefore, the medical staff person (especially, an unexperienced medical staff person) may frequently hesitate in judgement. As a result, under the above-mentioned circumstances, the medical staff person may frequently select either the lowest score of zero or the highest score of three. This results in a large error in score. In contrast, in this example, a question which is easy to judge is first asked and a question which is hard to judge is asked last, so that more accurate scoring is possible. In other words, even in a case of hesitating in determination (Yes in Step S104 in FIG. 2), there is only an error of one as the score in this example.

FIG. 16 is a flow chart for illustrating guidance and questions which are indicated by the indication unit 111 for an item of “5. Movement of Arms.”

First, in the Step S101 in FIG. 2,, the indication unit 111 uses the output device 140 to present the guidance of “Please carry out from a normal arm. Please start after a wrist and an elbow are raised in the air. Please tell “unexaminable” if examination cannot be performed.” Subsequently, in the Step S102 in FIG. 2, the indication unit 111 uses the output device 140 to present a question of “Against the gravity?” (Step S801).

Herein, it is assumed that the medical staff person judges that the arm of the patient is “against the gravity” (Yes in Step S801). In this case, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S801) to produce a recognized result thereof.

In this case, since this question is not the last question for this item (No in Step S105 of FIG. 2), the indication unit 111 returns to the Step S102 in FIG. 2 and then uses the output device 140 to present a question of “Does the arm fall to hit the bed?” (Step S802).

Herein, it is assumed that the medical staff person judges that the arm of the patient “falls to hit the bed” (Yes in Step S802). In this case, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S802) to produce a recognized result thereof.

In this event, since this question is the last question for this item (Yes in Step S105 in FIG. 2), the score determination unit 113 determines, based on the recognized result, a score for the item of “Movement of Arms” as two. Thereafter, the processing shifts to the next item (see Step S106 in FIG. 2).

On the other hand, in the above-mentioned Step S802, it is assumed that the medical staff person judges that the arm of the patient “does not fall to hit the bed” (No in Step S802). In this case, the medical staff person answers “no” with a speech from the microphone or touches the icon of “No” on the display screen of the display device with touch panel. As a result, in the Step S103 in FIG. 2, the reaction recognition unit 112 recognizes the speech of “yes” or touching of “Yes” (No in Step S802) to produce a recognized result thereof.

In this case, since this question is not the last question for this item (No in Step S105 of FIG. 2), the indication unit 111 returns to the Step S102 in FIG. 2 and then uses the output device 140 to present a question of “Keeping for ten seconds?” (Step S803).

Herein, it is assumed that the medical staff person judges that the patient “keeps for ten seconds” the arm (Yes in Step S803). In this case, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S803) to produce a recognized result thereof.

In this event, since this question is the last question for this item (Yes in Step S105 in FIG. 2), the score determination unit 113 determines, based on the recognized result, a score for the item of “Movement of Arms” as zero. Thereafter, the processing shifts to the next item (see Step S106 in FIG. 2).

On the other hand, in the above-mentioned Step S803, it is assumed that the medical staff person judges that the patient “does not keep for ten seconds” the arm (No in Step S803). In this case, the medical staff person answers “no” with a speech from the microphone or touches the icon of “No” on the display screen of the display device with touch panel. As a result, in the Step S103 in FIG. 2, the reaction recognition unit 112 recognizes the speech of “yes” or touching of “Yes” (No in Step S803) to produce a recognized result thereof.

In this event, since this question is the last question for this item (Yes of the Step S105 in FIG. 2), the score determination unit 113 determines, based on the recognized result, a score for the item of “Movement of Arms” as one. Thereafter, the processing shifts to the next item (see Step S106 in FIG. 2).

In the above-mentioned Step S801, it is assumed that the medical staff person judges that the arm of the patient is “not against the gravity” (No in Step S801). In this case, the medical staff person answers “no” with a speech from the microphone or touches the icon of “No” on the display screen of the display device with touch panel. As a result, in the Step S103 in FIG. 2, the reaction recognition unit 112 recognizes the speech of “no” or touching of “No” (Yes in Step S801) to produce a recognized result thereof.

In this case, since this question is not the last question for this item (No in Step S105 of FIG. 2), the indication unit 111 returns to the Step S102 in FIG. 2 and then uses the output device 140 to present a question of “Does not move at all?” (Step S804).

Herein, it is assumed that the medical staff person judges that the arm of the patient “does not move at all” (Yes in Step S804). In this case, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S804) to produce a recognized result thereof.

In this event, since this question is the last question for this item (Yes in Step S105 in FIG. 2), the score determination unit 113 determines, based on the recognized result, a score for the item of “Movement of Arms” as four. Thereafter, the processing shifts to the next item (see Step S106 in FIG. 2).

On the other hand, in the above-mentioned Step S804, it is assumed that the medical staff person judges that the arm of the patient “does not completely immobile” (No in Step S804). In this case, the medical staff person answers “no” with a speech from the microphone or touches the icon of “No” on the display screen of the display device with touch panel. As a result, in the Step S103 in FIG. 2, the reaction recognition unit 112 recognizes the speech of “no” or touching of “No” (No in Step S804) to produce a recognized result thereof.

In this event, since this question is the last question for this item (Yes in Step S105 in FIG. 2), the score determination unit 113 determines, based on the recognized result, a score for the item of “Movement of Arms” as three. Thereafter, the processing shifts to the next item (see Step S106 in FIG. 2).

On the other hand, in the above-mentioned Step S803, it is assumed that the medical staff person hesitates in determination, so that the elapsed time interval exceeds the allowed time interval (Yes in Step S104 in FIG. 2). In this event, the score determination unit 113 selects and produces, as the determined score, a higher score as a reaction of the medical staff person to the question at this time instant (Step 107 in FIG. 2), in of this example, since the reaction of “No” has a score higher than that of the reaction of “Yes”, the score determination unit 113 selects and produces one as a score for the item of “Movement of Arms.”

In addition, in the above-mentioned Step S804, it is assumed that the medical staff person hesitate in determination, so that the elapsed time interval exceeds the allowed time interval (Yes in Step S104 in FIG. 2). In this event, the score determination unit 113 selects and produces, as the determined score, a higher score as a reaction of the medical staff person to the question at this time instant (Step 107 in FIG. 2). In this example, since the reaction of “Yes” has a score higher than that of the reaction of “No”, the score determination unit 113 selects and produces four as a score for the item of “Movement of Arms.”

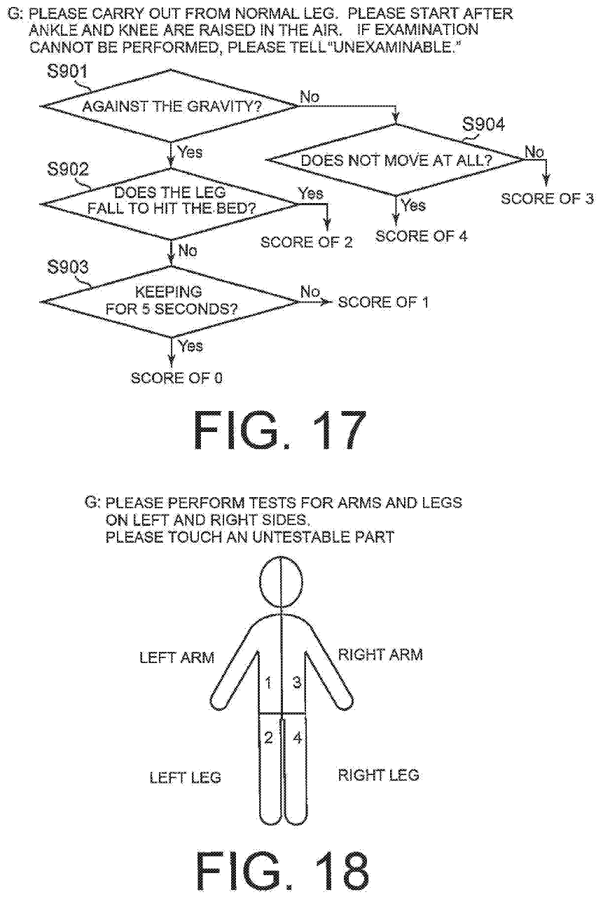

FIG. 17 is a flow chart for illustrating guidance and questions which are indicated by the indication unit 111 for an item of “6. Movement of Legs.”

First, in the Step S101 in FIG. 2, the indication unit 111 uses the output device 140 to present the guidance of “Please carry out from a normal leg. Please start after a wrist and an elbow are raised in the air. Please tell “unexaminable” if examination cannot be performed.” Subsequently, in the Step S102 in FIG. 2, the indication unit 111 uses the output device 140 to present a question of “Against the Gravity?” (Step S801).

Herein, it is assumed that the medical staff person judges that the leg of the patient is “against the gravity” (Yes in Step S901). In this case, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S901) to produce a recognized result thereof.

In this case, since this question is not the last question for this item (No in Step S105 of FIG. 2), the indication unit 111 returns to the Step S102 in FIG. 2 and then uses the output device 140 to present a question of “Does the leg fall to hit the bed?” (Step S902).

Herein, it is assumed that the medical staff person judges that the leg of the patient “falls to hit the bed” (Yes in Step S902). In this case, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S902) to produce a recognized result thereof.

In this event, since this question is the last question for this item (Yes in Step S105 in FIG. 2), the score determination unit 113 determines, based on the recognized result, a score for the item of “Movement of Legs” as two. Thereafter, the processing shifts to the next item (see Step S106 in FIG. 2).

On the other hand, in the above-mentioned Step S902, it is assumed that the medical staff person judges that the leg of the patient “does not fall to hit the bed” (No in Step S902). In this case, the medical staff person answers “no” with a speech from the microphone or touches the icon of “No” on the display screen of the display device with touch panel. As a result, in the Step S103 in FIG. 2, the reaction recognition unit 112 recognizes the speech of “yes” or touching of “Yes” (No in Step S902) to produce a recognized result thereof.

In this case, since this question is not the last question for this item (No in Step S105 of FIG. 2), the indication unit 111 returns to the Step S102 in FIG. 2 and then uses the output device 140 to present a question of “Keeping for five seconds?” (Step S903).

Herein, it is assumed that the medical staff person judges that the patient “keeps for five seconds” the leg (Yes in Step S903). In this case, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S903) to produce a recognized result thereof.

In this event, since this question is the last question for this item (Yes in Step S105 in FIG. 2), the score determination unit 113 determines, based on the recognized result, a score for the item of “Movement of Legs” as zero. Thereafter, the processing shifts to the next item (see Step S106 in FIG. 2).

On the other hand, in the above-mentioned Step S903, it is assumed that the medical staff person judges that the patient “does not keep for five seconds” the leg (No in Step S903). In this case, the medical staff person answers “no” with a speech from the microphone or touches the icon of “No” on the display screen of the display device with touch panel. As a result, in the Step S103 in FIG. 2, the reaction recognition unit 112 recognizes the speech of “no” or touching of “No” (No in Step S903) to produce a recognized result thereof.

In this event, since this question is the last question for this item (Yes in Step S105 in FIG. 2), the score determination unit 113 determines, based on the recognized result, a score for the item of “Movement of Legs” as one. Thereafter, the processing shifts to the next item (see Step S106 in FIG. 2).

In the above-mentioned Step S901, it is assumed that the medical staff person judges that the leg of the patient “is not against the gravity” (No in Step S901). In this case, the medical staff person answers “no” with a speech from the microphone or touches the icon of “No” on the display screen of the display device with touch panel. As a result, in the Step S103 in FIG. 2, the reaction recognition unit 112 recognizes the speed) of “no” or touching of “No” (No in Step S901) to produce a recognized result thereof.

In this case, since this question is not the last question for this item (No in Step S105 of FIG. 2), the indication unit 111 returns to the Step S102 in FIG. 2 and then uses the output device 140 to present a question of “Does not move at all?” (Step S904).

Herein, it is assumed that the medical staff person judges that the leg of the patient “does not move at all” (Yes in Step S904). In this case, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S904) to produce a recognized result thereof.

In this event, since this question is the last question for this item (Yes in Step S105 in FIG. 2), the score determination unit 113 determines, based on the recognized result, a score for the item of “Movement of Legs” as four. Thereafter, the processing shifts to the next item (see Step S106 in FIG. 2).

On the other hand, in the above-mentioned Step S904, it is assumed that the medical staff person judges that the leg of the patient “does not completely immobile” (No in Step S904). In this case, the medical staff person answers “no” with a speech from the microphone or touches the icon of “No” on the display screen of the display device with touch panel. As a result, in the Step S103 in FIG. 2, the reaction recognition unit 112 recognizes the speech of “no” or touching of “No” (No in Step S904) to produce a recognized result thereof.

In this event, since this question is the last question for this item (Yes in Step S105 in FIG. 2), the score determination unit 113 determines, based on the recognized result, a score for the item of “Movement of Legs” as three. Thereafter, the processing shifts to the next item (see Step S106 in FIG. 2).

On the other hand, in the above-mentioned Step S903, it is assumed that the medical staff person hesitates in determination, so that the elapsed time interval exceeds the allowed time interval (Yes in Step S104 in FIG. 2). In this event, the score determination unit 113 selects and produces, as the determined score, a higher score as a reaction of the medical staff person to the question at this time instant (Step 107 in FIG. 2). In this example, since the reaction of “No” has a score higher than that of the reaction of “Yes”, the score determination unit 113 selects and produces one as a score for the item of “Movement of Legs.”

In addition, in the above-mentioned Step S904, it is assumed that the medical staff person hesitates in determination, so that the elapsed time interval exceeds the allowed time interval (Yes in Step S104 in FIG. 2). In this event, the score determination unit 113 selects and produces, as the determined score, a higher score as a reaction of the medical staff person to the question at this time instant (Step 107 in FIG. 2). In this example, since the reaction of “Yes” has a score higher than that of the reaction of “No”, the score determination unit 113 selects and produces four as a score for the item of “Movement of Arms.”

FIG. 18 is a flow chart for illustrating guidance and a question which are indicated by the indication unit 111 for an item of “7. Motor Ataxia” and an example of display on the display screen of the display device with touch panel.

First, in the Steps S101 and S102 in FIG. 2, the indication unit 111 uses the output device 140 to provide guidance and ask a question of “Please perform tests for arms and legs on left and right sides. Please touch an untestable part.” In a case of this item, on the display screen of the display device with touch panel, an image in which the numbers of 1 to 4 are written for a left arm, a left leg, a right arm, and a right leg is displayed as shown in FIG. 18.

The medical staff person observes the patient and touches the untestable part of the displayed image. The reaction recognition unit 112 produces a touch signal corresponding to the touched part as the recognized result (Step S103 in FIG. 2). When a touch operation of the medical staff person finishes, this touch operation corresponds to the last question for this item (Yes in Step S105 of FIG. 2). Therefore, the score determination unit 113 automatically calculates and produces a score based on the touch signal (the recognized result).

On the other hand, it is assumed that the medical staff person hesitates in determination, so that the elapsed time interval exceeds the allowed time interval (Yes in Step S104 in FIG. 2). In this event, the score determination unit 113 selects and produces, as the determined score, a highest score as a reaction of the medical staff person to the question at this time instant (Step 107 in FIG. 2).

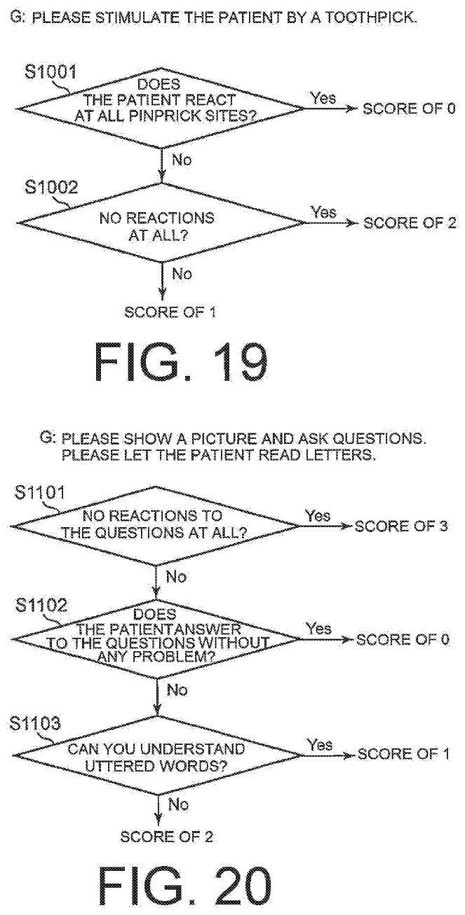

FIG. 19 is a flow chart for illustrating guidance and questions which are indicated by the indication unit 111 for an item of “8. Sensory.”

First, in the Step S101 in FIG. 2, the indication unit 111 uses the output device 140 to present the guidance of “Please stimulate the patient by a toothpick.” Subsequently, in the Step S102 in FIG. 2, the indication unit 111 uses the output device 140 to present a question of “Does the patient react at all pinprick sites?” (Step S1001). Herein, if the patient reacts to all stimuli, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S1001) to produce a recognized result thereof.

In this event, since this question is the last question for this item (Yes in Step S105 in FIG. 2), the score determination unit 113 determines, based on the recognized result, a score for the item of “Sensory” as zero. Thereafter, the processing shifts to the next item (see Step S106 in FIG. 2).

On the other hand, in the above-mentioned Step S1001, if the patient does not react to all stimuli, the medical staff person answers “no” with a speech from the microphone or touches the icon of “No” on the display screen of the display device with touch panel.

In this case, since this question is not the last question for this item (No in Step S105 of FIG. 2), the indication unit 111 presents a question of “No reaction at all?” (Step S102 in FIG. 2. and Step S1002 in FIG. 19). Herein, if the patient has no reaction to the stimuli at all, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S1002) to produce a recognized result thereof.

In this event, since this question is the last question for this item (Yes in Step S105 in FIG. 2), the score determination unit 113 determines, based on the recognized result, a score for the item of “Sensory” as two. Thereafter, the processing shifts to the next item (see Step S106 in FIG. 2).

On the other hand, in the above-mentioned Step S1002, unless the patient has no reaction at all, the medical staff person answers “yes” with a speech from the microphone or touches the icon of “No” on the display screen of the display device with touch panel.

In this event, since this question is the last question for this item (Yes in Step S105 in FIG. 2), the score determination unit 113 determines, based on the recognized result, a score for the item of “Sensory” as one. Thereafter, the processing shifts to the next item (see Step S106 in FIG. 2).

On the other hand, in the above-mentioned Step S1002, it is assumed that the medical staff person hesitates in determination, so that the elapsed time interval exceeds the allowed time interval (Yes in Step S104 in FIG. 2). In this event, the score determination unit 113 selects and produces, as the determined score, a higher score as a reaction of the medical staff person to the question at this time instant (Step 107 in FIG. 2), In this example, since the reaction of “Yes” has a score higher than that of the reaction of “No”, the score determination unit 113 selects and produces two as a score for the item of “Sensory.”

FIG. 20 is a flow chart for illustrating guidance and questions which are indicated by the indication unit 111 for an item of “9. Best Language.”

First, in the Step S101 in FIG. 2, the indication unit 111 uses the output device 140 to present the guidance of “Please show a picture and ask questions. Please let the patient read letters.” Subsequently, in the Step S102 in FIG. 2, the indication unit 111 uses the output device 140 to present a question of “No reaction to the questions at all?” (Step S1101). Herein, if the patient has no reaction to the questions at all, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S1101) to produce a recognized result thereof.

In this event, since this question is the last question for this item (Yes in Step S105 in FIG. 2), the score determination unit 113 determines, based on the recognized result, a score for the item of “Best Language” as three. Thereafter, the processing shifts to the next item (see Step S106 in FIG. 2).

On the other hand, in the above-mentioned Step S1101, if the patient has any reaction to the questions, the medical staff person answers “no” with a speech from the microphone or touches the icon of “No” on the display screen of the display device with touch panel.

In this case, since this question is not the last question for this item (No in Step S105 of FIG. 2), the indication unit 111 presents a question of “Does the patient answer to the questions without any problem?” (Step S102 in FIG. 2 and Step S1102 in FIG. 20). Herein, if the patient answers to the questions without any problem, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S1102) to produce a recognized result thereof.

In this event, since this question is the last question for this item (Yes in Step S105 in FIG. 2), the score determination unit 113 determines, based on the recognized result, a score for the item of “Best Language” as zero. Thereafter, the processing shifts to the next item (see Step S106 in FIG. 2).

On the other hand, in the above-mentioned Step S110, unless the patient answers to the questions without any problem, the medical staff person answers “no” with a speech from the microphone or touches the icon of “No” on the display screen of the display device with touch panel.

In this case, since this question is not the last question for this item (No in Step S105 of FIG. 2), the indication unit 111 presents a question of “Can the patient understand uttered words?” (Step S102 in FIG. 2 and Step S1103 in FIG. 20). Herein, if the medical staff person judges that the patient can understand the words, the medical staff person answers “yes” with a speech from the Microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S1103) to produce a recognized result thereof.

In this event, since this question is the last question for this item (Yes in Step S105 in FIG. 2), the score determination unit 113 determines, based on the recognized result, a score for the item of “Best Language” as one. Thereafter, the processing shifts to the next item (see Step S106 in FIG. 2).

On the other hand, in the above-mentioned Step S1103, if the medical staff person judges that the patient cannot understand the words, the medical staff person answers “no” with a speech from the microphone or touches the icon of “No” on the display screen of the display device with touch panel. In this event, since this question is the last question for this item (Yes in Step S105 in FIG. 2), the score determination unit 113 determines, based on the recognized result, a score for the item of “Best Language” as two. Thereafter, the processing shifts to the next item (see Step S106 in FIG. 2).

On the other hand, in the above-mentioned Step S1103, it is assumed that the medical staff person hesitates in determination, so that the elapsed time interval exceeds the allowed time interval (Yes in Step S104 in FIG. 2). In this event, the score determination unit 113 selects and produces, as the determined score, a higher score as a reaction of the medical staff person to the question at this time instant (Step 107 in FIG. 2). In this example, since the reaction of “No” has a score higher than that of the reaction of “Yes”, the score determination unit 113 selects and produces two as a score for the item of “Best Language.”

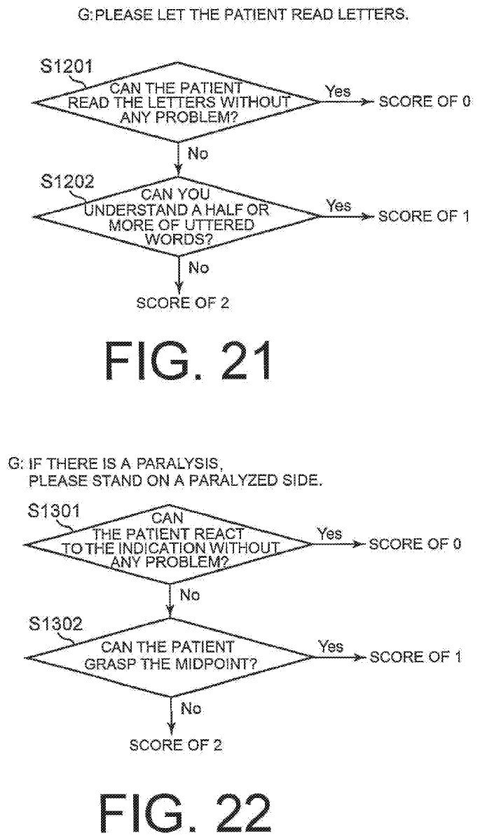

FIG. 21 is a flow chart for illustrating guidance and questions which are indicated by the indication unit 111 for an item of “10. Dysarthria.”

First, in the Step S101 in FIG. 2, the indication unit 111 uses the output device 140 to present the guidance of “Please let the patient read letters.” Subsequently, in the Step S102 in FIG. 2, the indication unit 111 uses the output device 140 to present a question of “Can the patient read the letters without any problem?” (Step S1201). Herein, if the patient can read all of the letters without any problem, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S1201) to produce a recognized result thereof.

In this event, since this question is the last question for this item (Yes in Step S105 in FIG. 2), the score determination unit 113 determines, based on the recognized result, a score for the item of “Dysarthria” as zero. Thereafter, the processing shifts to the next item (see Step S106 in FIG. 2).

On the other hand, in the above-mentioned step S1201, if the patient cannot read all of the letters without any problem, the medical staff person answers “no” with a speech from the microphone or touches the icon of “No” on the display screen of the display device with touch panel.

In this case, since this question is not the last question for this item (No in Step S105 of FIG. 2), the indication unit 111 presents a question of “Can you understand a half or more of uttered words?” (Step S102 in FIG. 2 and Step S1202 in FIG. 21). Herein, if the medical staff person can understand the uttered words of the patient, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S1103) to produce a recognized result thereof.

In this event, since this question is the last question for this item (Yes in Step S105 in FIG. 2), the score determination unit 113 determines, based on the recognized result, a score for the item of “Dysarthria” as one. Thereafter, the processing shifts to the next item (see Step S106 in FIG. 2).

On the other hand, in the above-mentioned Step S1202, if the medical staff person cannot understand the uttered words of the patient, the medical staff person answers “no” with a speech from the microphone or touches the icon of “No” on the display screen of the display device with touch panel.

In this event, since this question is the last question for this item (Yes in Step S105 in FIG. 2), the score determination unit 113 determines, based on the recognized result, a score for the item of “Dysarthria” as two. Thereafter, the processing shifts to the next item (see Step S106 in FIG. 2).

On the other hand, in the above-mentioned Step S1202, it is assumed that the medical staff person hesitates in determination, so that the elapsed time interval exceeds the allowed time interval (Yes in Step S104 in FIG. 2). In this event, the score determination unit 113 selects and produces, as the determined score, a higher score as a reaction of the medical staff person to the question at this time instant (Step 107 in FIG. 2). In this example, since the reaction of “No” has a score higher than that of the reaction of “Yes”, the score determination unit 113 selects and produces two as a score for the item of “Dysarthria.”

FIG. 22 is a flow chart for illustrating guidance and questions which are indicated by the indication unit 111 for an item of “11. Extinction and Inattention.”

First, in the Step S101 in FIG. 2, the indication unit 111 uses the output device 140 to present the guidance of “if there is a paralyzed side, please stand on a paralyzed side.” Subsequently, in the Step S102 in FIG. 2, the indication unit 111 uses the output device 140 to present a question of “Can the patient react to the indication without any problem?” (Step S1301). Herein, if the patient can react without any problem, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S1301) to produce a recognized result thereof.

In this event, since this question is the last question for this item (Yes in Step S105 in FIG. 2), the score determination unit 113 determines, based on the recognized result, a score for the item of “Extinction and Inattention” as zero. Thereafter, the processing shifts to the next item (see Step S106 in FIG. 2).

On the other hand, in the above-mentioned Step S1301, unless the patient can react without any problem, the medical staff person answers “no” with a speech from the microphone or touches the icon of “No” on the display screen of the display device with touch panel.

In this case, since this question is not the last question for this item (No in Step S105 of FIG. 2), the indication unit 111 presents a question of “Can the patient grasp the midpoint?” (Step S102 in FIG. 2 and Step S1302 in FIG. 22). Herein, if the patient can grasp the midpoint, the medical staff person answers “yes” with a speech from the microphone or touches the icon of “Yes” on the display screen of the display device with touch panel. As a result, in the Step S103 in FIG. 2, the reaction recognition unit 112 recognizes the speech of “yes” or touching of “Yes” (Yes in Step S1302) to produce a recognized result thereof.

In this event, since this question is the last question for this item (Yes in Step S105 in FIG. 2), the score determination unit 113 determines, based on the recognized result, a score for the item of “Extinction and Inattention” as one. Thereafter, the processing shifts to the next item (see Step S106 in FIG. 2).

On the other hand, in the above-mentioned Step S1302, if the patient cannot grasp the midpoint, the medical staff person answers “no” with a speech from the microphone or touches the icon of “No” on the display screen of the display device with touch panel.

In this event, since this question is the last question for this item (Yes in Step S105 in FIG. 2), the score determination unit 113 determines, based on the recognized result, a score for the item of “Extinction and Inattention” as two.

On the other hand, in the above-mentioned Step S1302, it is assumed that the medical staff person hesitates in determination, so that the elapsed time interval exceeds the allowed time interval (Yes in Step S104 in FIG. 2). In this event, the score determination unit 113 selects and produces, as the determined score, a higher score as a reaction of the medical staff person to the question at this time instant (Step 107 in FIG. 2). In this example, since the reaction of “No” has a score higher than that of the reaction of “Yes”, the score determination unit 113 selects and produces two as a score for the item of “Extinction and Inattention.”

The patient status determination devices 100 to 100C mentioned above may be implemented by hardware or may be implemented by software. Alternatively, the patient status determination devices 100 to 100C may be implemented by a combination of hardware and software.

It is noted that the program 122, which is readable by the data processing devices 110 to 110C, may be supplied to the patient status determination devices 100 to 100C in a state where it is non-transitorily stored in various types of recording media which is readable by a computer. Such recording media include, for example, a magnetic tape, a magnetic disk, a magneto-optical disc, a CD-ROM (Compact Disc-Read Only Memory), a CD-R (Compact Disc-Readable), a CD-RW (Compact Disc-ReWritable), and a semiconductor memory.

While the present invention has been described with reference to the example embodiments and the example thereof, the present invention is not limited thereto. For example, the present invention includes modes obtained by appropriately combining a part or a whole of the example embodiments and the example described so far or a mode obtained by appropriately modifying those modes.

For example, although the data processing device comprises the time measurement unit in any of the above-mentioned example embodiments, it may be omitted in the present invention, In this case, although the problem of the measurement time interval (determination time interval) may remain, it is possible to resolve the problem of the hesitation in decision on measurement (determination) of scoring. This is because the appropriate guidance and indication are given to the medical staff person.

In addition, a “help” button may be displayed on the display screen of the display device with touch panel. In this case, for example, when an unexperienced medical staff person pushes the “help” button, the “determined score” for an analogues patient in the past, that is scored by a skilled medical staff person in the past, is displayed on the display screen in association with the indicated item. Thus, the unexperienced medical staff person can perform appropriate scoring with reference to the displayed contents.

Furthermore, although the totalization device 200 merely receives the determined score from the patient status determination device and totalizes the score in the above-mentioned example embodiments, the present invention is not limited thereto. That is, the present invention may be applied to an electronic medical chart system comprising a medical recording database in lieu of the totalization device 200.

In this case, each of the patient status determination devices 100A to 100C wirelessly transmits, to the electronic medical chart system via the communication interface 150, the recorded information (indicated item, determined score, and elapsed time interval) recorded in the score and time recording unit 126 of the storage device 120A or 120B in association with the patient ID. The electronic medical chart system records (transcribes) the received recorded information (which is associated with the patient ID) together with the totalized result in a corresponding field of the patient ID on the medical recording database.

Furthermore, when the communication interface 150 comprises the connector, each of the patient status determination devices 100A to 100C writes, in an outside recording medium via the connector, the recorded information (indicated item, determined score, and elapsed time interval) recorded in the score and time recording unit 126 of the storage device 120A or 120B in association with the patient ID. Then, the electronic medical chart system reads the recorded information recorded in the outside recording medium via a connector provided in the electronic medical chart system, and records (transcribes) the read information, together with the totalized result, in the corresponding field of the patient ID on the medical recording database.

A part or a whole of the example embodiments described above may also be described as the following supplementary notes without being limited thereto.

(Supplementary Note 1)

A patient status determination device comprising:

an indication unit configured to sequentially indicate, to a medical staff person, a plurality of items to be measured for a patient;

a reaction recognition unit configured to recognize a reaction from the medical staff person to the indicated item to produce a recognized result; and

a score determination unit configured to determine, based on the recognized result, a score for the indicated item to produce a determined score,

wherein the indication unit is configured to present, to the medical staff person, each of the plurality of items in the form of at least one question associated with the item.

(Supplementary Note 2)

The patient status determination device according to Supplementary Note 1, wherein the indication unit comprises a speech notification means configured to notify the medical staff person of each of the question corresponding to the indicated item with a speech.

(Supplementary Note 3)

The patient status determination device according to Supplementary Note 1 or 2, wherein the reaction recognition unit comprises a speech identification means configured to receive an answer speech uttered from the medical staff person as the reaction and to identify the answer speech to produce an identified result as the recognized result.

(Supplementary Note 4)

The patient status determination device according to any one of Supplementary Notes 1 to 3, further comprising a time measurement unit configured to measure an elapsed time interval from a starting time instant when indication of each item to the medical staff person is started.

(Supplementary Note 5)

The patient status determination device according to Supplementary Note 4,

wherein an allowed time interval is preliminarily set for each of the plurality of items, and

wherein the score determination unit is configured to select and produce as the determined score, in a case where the reaction cannot be obtained from the medical staff person at a time instant when the elapsed time interval exceeds the allowed time interval, a higher score as a reaction to the question at the time instant.

(Supplementary Note 6)

The patient status determination device according to Supplementary Note 4, further comprising a score and time recording unit configured to record, as recorded information, the determined score and the elapsed time interval in association with the indicated item.

(Supplementary Note 7)

The patient status determination device according to Supplementary Note 6, wherein the indication unit is configured to change, based on the recorded information recorded in the score and time recording unit during past measurements including at least the last time, notification contents to be indicated to the medical staff person in a current measurement.

(Supplementary Note 8)

The patient status determination device according to Supplementary Note 7, wherein the changed notification contents include the allowed time interval which is set for the indicated item.

(Supplementary Note 9)

The patient status determination device according to Supplementary Note 6, further comprising a measurement state recording unit configured to record a measurement state of the patient.

(Supplementary Note 10)

The patient status determination device according to Supplementary Note 9, wherein the indication unit is configured to change, based on the recorded information recorded in the score and time recording unit and the measurement state recorded in the measurement state recording unit during past measurements including at least the last time, notification contents to be indicated to the medical staff person in a current measurement.

(Supplementary Note 11)

The patient status determination device according to Supplementary Note 10, wherein the changed notification contents include the allowed time interval which is set for the indicated item.

(Supplementary Note 12)

The patient status determination device according to Supplementary Note 4, wherein an allowed time interval is preliminarily set for each of the plurality of items, wherein the patient status determination device further comprises an analogous case extraction unit which is configured to extract, when the reaction cannot be obtained from the medical staff person at a time instant when the elapsed time interval exceeds the allowed time interval, one analogous case from an analogous case database for preliminarily saving a plurality of analogous cases, the one analogous case being analogous to a status of the patient, and to present the extracted analogous case to the medical staff person.

(Supplementary Note 13)

The patient status determination device according to any one of Supplementary Notes 1 to 12, wherein the plurality of items include a specific item having a plurality of questions,
wherein the indication unit is configured to:

indicate, as a first one of the plurality of questions for the specific item, a question for which the medical staff person can easily judge the status of the patient; and

indicate, as a last one of the plurality of questions for the specific item, a question for which the medical staff person can hardly judge the status of the patient.

(Supplementary Note 14)

The patient status determination device according to any one of Supplementary Notes 1 to 13, wherein the plurality of items comprise items which are defined by NIHSS (National Institute of Health Stroke Scale).

(Supplementary Note 15)

A patient status determination system comprising:

the patient status determination device according to any one of Supplementary Notes 1 to 14; and

a totalization device configured to receive the determined score from the patient status determination device to totalize the score.

(Supplementary Note 16)

A patient status determination method comprising:

sequentially indicating, by an indication unit, to a medical staff person, a plurality of items to be measured for a patient;

recognizing, by a reaction recognition unit, a reaction from the medical staff person to the indicated item to produce a recognized result; and

determining, by a score determination unit, based on the recognized result, a score for the indicated item to produce a determined score,

wherein the indication unit is configured to present, to the medical staff person, each of the plurality of items in the form of at least one question associated with the item.

(Supplementary Note 17)

A recording medium recording a patient status determination program which causes a computer to execute:

an indication step of sequentially indicating, to a medical staff person, a plurality of items to he measured for a patient;

a reaction recognition step of recognizing a reaction from the medical staff person to the indicated item to produce a recognized result; and

a score determination step of determining, based on the recognized result, a score for the indicated item to produce a determined score,

wherein the indication step causes the computer to present, to the medical staff person, each of the plurality of items in the form of at least one question associated with the item.

This application is based upon and claims the benefit of priority from Japanese patent application No. 2017-235687, filed on Dec. 8, 2017, the disclosure of which is incorporated herein in its entirety by reference.

### REFERENCE SIGNS LIST

100, 100A, 100B, 100C patient status determination device
110, 110A, 110B, 110C date processing device
111, 111A, 111B indication unit
112 reaction recognition unit
113 score determination unit
114 time measurement unit
115 analogous example extraction unit
118, 118A, 118B, 118C control unit
120, 120A, 120B storage device
122 program
124 item storage unit
126 score and time recording unit
128 measurement state recording unit
130 input device
140 output device
150 communication interface
200 totalization device
210 data processing device
212 totalization unit
220 storage device
230 communication interface

1. A patient status determination device comprising:
an indicator configured to sequentially indicate, to a medical staff person, a plurality of items to be measured for a patient;
a reaction recognizer configured to recognize a reaction from the medical staff person to the indicated item to produce a recognized result; and
a score determiner configured to determine, based on the recognized result, a score for the indicated item to produce a determined score,
wherein the indicator is configured to present, to the medical staff person, each of the plurality of items in the form of at least one question associated with the item.

2. The patient status determination device as claimed in claim 1, wherein the indicator comprises a speech notifier configured to notify the medical staff person of each of the question corresponding to the indicated item with a speech.

3. The patient status determination device as claimed in claim 1, wherein the reaction recognizer comprises a speech identifier configured to receive an answer speech uttered from the medical staff person as the reaction and to identify the answer speech to produce an identified result as the recognized result.

4. The patient status determination device as claimed in claim 1, further comprising a time measurer configured to measure an elapsed time interval from a starting time instant when indication of each item to the medical staff person is started.

5. The patient status determination device as claimed in claim 4, wherein an allowed time interval is preliminarily set for each of the plurality of items, and
wherein the score determiner is configured to select and produce as the determined score, in a case where the reaction cannot be obtained from the medical staff person at a time instant when the elapsed time interval exceeds the allowed time interval, a higher score as a reaction to the question at the time instant.

6. The patient status determination device as claimed in claim 4, further comprising a score and time recorder configured to record, as recorded information, the determined score and the elapsed time interval in association with the indicated item.

7. The patient status determination device as claimed in claim 6, wherein the indicator is configured to change, based on the recorded information recorded in the score and time recorder during past measurements including at least the last time, notification contents to be indicated to the medical staff person in a current measurement.

8. The patient status determination device as claimed in claim 7, wherein the changed notification contents include the allowed time interval which is set for the indicated item.

9. The patient status determination device as claimed in claim 6, further comprising a measurement state recorder configured to record a measurement state of the patient.

10. The patient status determination device as claimed in claim 9, wherein the indicator is configured to change, based on the recorded information recorded in the score and time recorder and the measurement state recorded in the measurement state recorder during past measurements including at least the last time, notification contents to be indicated to the medical staff person in a current measurement.

11. The patient status determination device as claimed in claim 10, wherein the changed notification contents include the allowed time interval which is set for the indicated item.

12. The patient status determination device as claimed in claim 4, wherein an allowed time interval is preliminarily set for each of the plurality of items,
wherein the patient status determination device further comprises an analogous case extractor which is configured to extract, when the reaction cannot be obtained from the medical staff person at a time instant when the elapsed time interval exceeds the allowed time interval, one analogous case from an analogous case database for preliminarily saving a plurality of analogous cases, the one analogous case being analogous to a status of the patient, and to present the extracted analogous case to the medical staff person.

13. The patient status determination device as claimed in claim 1, wherein the plurality of items include a specific item having a plurality of questions,
wherein the indicator is configured to:
indicate, as a first one of the plurality of questions for the specific item, a question for which the medical staff person can easily judge the status of the patient; and
indicate, as a last one of the plurality of questions for the specific item, a question for which the medical staff person can hardly judge the status of the patient.

14. The patient status determination device as claimed in claim 1, wherein the plurality of items comprise items which are defined by NIHSS (National Institute of Health Stroke Scale).

15. A patient status determination system comprising:
the patient status determination device described in claim 1; and
a totalization device configured to receive the determined score from the patient status determination device to totalize the score.

16. A patient status determination method comprising:
sequentially indicating, to a medical staff person, a plurality of items to be measured for a patient;
recognizing a reaction from the medical staff person to the indicated item to produce a recognized result; and
determining, based on the recognized result, a score for the indicated item to produce a determined score,
wherein the indicating presents, to the medical staff person, each of the plurality of items in the form of at least one question associated with the item.

17. A non-transitory recording medium recording a patient status determination program which causes a computer to execute:
an indication step of sequentially indicating, to a medical staff person, a plurality of items to be measured for a patient;
a reaction recognition step of recognizing a reaction from the medical staff person to the indicated item to produce a recognized result; and
a score determination step of determining, based on the recognized result, a score for the indicated item to produce a determined score,
wherein the indication step causes the computer to present, to the medical staff person, each of the plurality of items in the form of at least one question associated with the item.
